(19) United States
(12) Patent Application Publication
(10) Pub. No.: US 2005/0032837 A1
(43) Pub. Date: Feb. 10, 2005

(54) 4-Phenyl-piperidine compounds and their use as modulators of opioid receptors

(75) Inventors: Stanton McHardy, Coventry, RI (US); Spiros Liras, Stonington, CT (US); Sara Guediche, Pittsburgh, PA (US); Jotham W. Coe, Niantic, CT (US)

Correspondence Address: SCULLY SCOTT MURPHY & PRESSER, PC, 400 GARDEN CITY PLAZA, GARDEN CITY, NY, 11530, US

(73) Assignee: Pfizer, Inc., New York, NY (US)

(21) Appl. No.: 10/823,026

(22) Filed: Apr. 13, 2004

Related U.S. Application Data
(60) Provisional application No. 60/462,651, filed on Apr. 14, 2003.

(51) Int. Cl. C07D 211/32; C07D 211/54; A61K 31/445

(52) U.S. Cl. 514/317; 546/233; 546/216

(57) ABSTRACT

The subject invention relates to 4-phenyl-piperidine derivatives, pharmaceutical compositions comprising such derivatives and methods of using such derivatives to treat disease states, disorders and conditions mediated by an opioid receptor. The subject also particularly relates to using such derivatives to treat certain disease states, disorders and conditions, for example irritable bowel syndrome, drug addiction or dependency, including alcohol addiction, depression, anxiety, schizophrenia, anxiety, schizophrenia and eating disorders, among numerous other disease states, disorders and conditions as more fully described herein.

### FIELD OF THE INVENTION

The subject invention relates to 4-phenyl-piperidine derivatives, pharmaceutical compositions comprising such derivatives and methods of using such derivatives to treat disease states, disorders and conditions mediated by an opioid receptor. The subject also particularly relates to using such derivatives to treat certain disease states, disorders and conditions, for example irritable bowel syndrome, drug addiction, including alcohol addiction, abuses or dependency, depression, anxiety, schizophrenia and eating disorders, among numerous other disease states, disorders and conditions as more fully described herein.

### BACKGROUND OF THE INVENTION

The compounds of the present invention bind to opioid receptors (e.g. delta, kappa and mu opioid receptors). Compounds that bind to such receptors are useful in the treatment of diseases modulated by opioid receptors, for example irritable bowel syndrome; constipation; nausea; vomiting; and pruritic dermatoses, such as allergic dermatitis and atopy in animals and humans. Compounds that bind to opioid receptors have also been indicated in the treatment of eating disorders, opioid overdoses, depression, anxiety, schizophrenia, smoking and alcohol addiction and dependence, sexual dysfunction, shock, stroke, spinal damage and head trauma, among others.

Certain 4-arylpiperidine-based compounds are disclosed in European patent applications EP 287339, EP 506468 and EP 506478 as opioid agents (antagonists/agonists or the like). In addition, International Patent Application WO 95/15327 discloses azabicycloalkane derivatives useful as neuroleptic agents. 3-Azabicyclo[3.1.0] hexane derivatives useful as opioid receptor agents are also disclosed in WO 00/39089.

A number of the prior art compounds, although active as opioid receptor modulators, are readily metabolized by the enzyme CYP2D6, resulting in variability in the pharmacokinetics of an administered medicine from patient to patient. It is furthermore beneficial to obtain medicines, for example medicines that bind to opioid receptors that are not substrates of the enzyme CYP2D6. The presence of CYP2D6 enzyme among the human population is variable, and therefore it is easier to develop dosage schemes for a medicine that are more generally applicable to a human population if the medicine is not metabolized by CYP2D6.

### SUMMARY OF THE INVENTION

The subject invention provides a compound of the formula I, 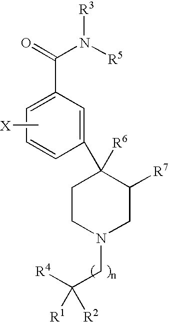
wherein X is H, halogen, or CN;
R¹ and R² are independently H, C₁-C₆ alkyl, —(CH₂)ₖ-aryl, —(CH₂)ₖ-heteroaryl, wherein said alkyl, —(CH₂)ₖ-aryl or —(CH₂)ₖ-heteroaryl group is optionally substituted anywhere on said group with one or more R¹² groups, or, with the carbon to which R¹ and R² are attached, are connected to form a C₃-C₇ cycloalkyl or a 4-7 membered carbocyclic or heterocycloalkyl comprising from one to three hetero moities selected from O, S, —C(═O), and N; and wherein said cycloalkyl or heterocycloalkyl optionally contains one or more double bonds; and wherein said cycloalkyl or heterocycloalkyl is optionally fused to or substituted with a C₆-C₁₄ aryl or 5-14 membered heteroaryl group; wherein said C₃-C₇ cycloalkyl or 4-7 membered carbocyclic or heterocycloalkyl formed by R¹ and R² can each optionally be substituted by from one to three R¹² groups, and said optionally fused or substituted aryl or heteroaryl, substituted alkyl, substituted aryl optionally fused aryl or heteroaryl may each optionally independently be substituted with from one to six R¹² groups in any stereochemical relationship; wherein the R¹² groups are independently selected from H, R¹³, R¹⁶, —C₁-C₄ alkyl optionally containing one or two unsaturated bonds, halogen, —OR¹³, —NO₂, —CN, —C₃-C₆ cycloalkyl, aryl, substituted aryl, wherein said aryl or substituted aryl is independently optionally substituted with 1-3 R¹⁸ groups, —C(R⁴)(C₁-C₄ alkyl)(C₁-C₄ alkyl) wherein said alkyl groups may form a C₃-C₇ carbocyclic ring, —(CH₂)ᵥ—NR¹³R¹⁴, —NR¹³C(═O)R¹⁴, —C(═O)NR¹³R¹⁴, —OC(═O)R¹³, —C(═O)OR¹³, —C(═O)R¹³, —NR¹³C(═O)OR¹⁴, —NR¹³C(═O)NR¹⁴R¹⁵, —NR¹³S(═O)₂R, —NR¹⁷S(═O)₂NR¹³R¹⁴ and —S(═O)₂R¹³;
R¹⁵ is H, F, Cl, —OH, —C₁-C₄ alkyl, —C≡N, —NR¹³C(═O)R¹⁴, —C(═O)NR¹³R¹⁴, —O(C₁-C₄)alkyl, —NH(C₁-C₄ alkyl), —N(C₁-C₄ alkyl)(C₁-C₄ alkyl), —(CH₂)ₙOH, —(CH₂)ₙ—C≡N, —(CH₂)ₙ—NR¹³C(═O)R¹⁴, (CH₂)ₙ—C(═O)NR¹³R¹⁴, —(CH₂)ₙ—O(C₁-C₄)alkyl, —(CH₂)ₙ—NH₂, —(CH₂)ₙ—NH(C₁-C₄ alkyl) or —(CH₂)ₙ—N(C₁-C₄ alkyl)(C₁-C₄ alkyl);
R⁴ is absent or is H, —C₁-C₄ alkyl which may optionally contain one or two unsaturated bonds, —OH, O—(C₁-C₄)alkyl, (C₁-C₄)-alkyl-OH, (CH₂)ₙNH₂, —(CH₂)ₙ—NH(C₁-C₄ alkyl), (CH₂)ₙ—N(C₁-C₄)alkyl(C₁-C₄)alkyl, —(CH₂)ₙ—NHC(═O)(C₁-C₄ alkyl), —(CH₂)ₙ—NO₂, —(CH₂)ₙ—C≡N, —(CH₂)ₙ—C(═O)NH₂, —(CH₂)ₙ—C(═O)NH(C₁-C₄ alkyl) or —(CH₂)ₙ—C(═O)N(C₁-C₄ alkyl)(C₁-C₄ alkyl), CN, NO₂, —OR¹⁶;
R³ and R⁵ are independently H, alkyl C₁-C₆, substituted alkyl C₁-C₆, cycloalkyl C₁-C₆ and substituted cycloalkyl C₁-C₆, (C₂-C₄)alkyl-O—(C₁-C₄)alkyl, (C₂-C₄)alkyl-NH(C₁-C₄ alkyl), (C₂-C₄)alkyl-N(C₁-C₄ alkyl)(C₁-C₄ alkyl), (C₁-C₄)alkyl-heterocyclic;
R⁶ and R⁷ are independently C₁-C₄ alkyl, more preferably methyl;
each R¹³, R¹⁴, and R¹⁵ are independently selected from H, —C₁-C₄ alkyl, —(C₂-C₄ alkyl)-O—(C₁-C₄-alkyl), —(CH₂)ᵥ—NR¹⁶R¹⁷, or a 4- to 7-membered heterocyclic group; or R¹³ and R¹⁴ when in —NR¹³R¹⁴, may optionally be connected to form a 4 to 6 membered heterocyclic group, which heterocyclic group optionally comprises from 1 to 3 further hetero moieties selected from N, S, O and —C(═O);
R¹⁶ and R¹⁷ are independently H, C₁-C₆ alkyl or together may form a 4- to 7-membered heterocyclic group;
k is an integer selected from zero, 1, 2, 3, 4, and 5; and
v is an integer selected from 2, 3, 4, and 5; and
n is an integer selected from zero, 1, 2, 3, 4, and 5;
and pharmaceutically acceptable salts thereof;
with the proviso that;
   (a) in said 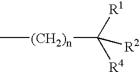
   group, when n is 0, R¹, R² or R⁴ cannot be a heteroatom or contain a heteroatom which is directly linked to the carbon of said
   (b) 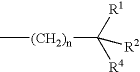
   group when said carbon is sp³ hybridized; and
R¹³ and R¹⁴ cannot be H in a —NHS(═O)₂R¹⁴ or a —SO₂R¹³ group.

Preferred compounds according to the present invention may be favorably represented by the following chemical structure II: 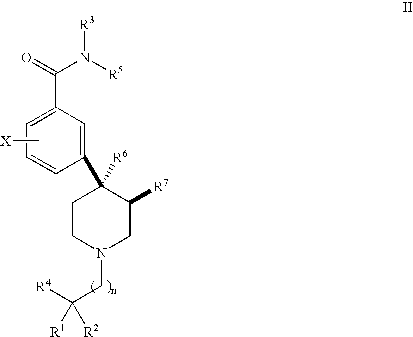
where each of the substituents is represented as described above and the preferred relative stereochemistry between R⁶ and R⁷ is trans. The present invention also relates to enantiomers of the trans diastereomeric compound of formula II depicted above.

In certain preferred aspects of the present invention, R³ and R⁵ are H. In other preferred aspects of the present invention, X is hydrogen. In other preferred aspects, R⁶ and R⁷ are CH₃. In other aspects of the present invention, n is preferably 1, 2 or 3, even more preferably 1. In still other preferred aspects of the present invention, R⁴ is OH, CH₂OH, NH₂. NHCOCH₃ or CN, even more preferably OH. In other preferred aspects of the invention, R¹ and R², together with the carbon to which they are attached, form a carbocyclic group fused to a phenyl ring (phenyl-fused), even more preferably an indane ring system, or an unsubstituted or substituted carbocyclic group, more preferably a cyclobutane, cyclopentane or cyclohexane group, even more preferably a cyclobutane group which is substituted with an unsubstituted phenyl group or a phenyl group substituted with one or more R¹² groups.

In other preferred embodiments, the following compounds and their pharmaceutically acceptable salts are also preferred:
(+/−)-3-(trans-3,4-Dimethyl-1-phenethyl-piperidin-4-yl)-benzamide;
(+/−)-3-(1-Indan-2-ylmethyl-trans-3,4-dimethyl-piperidin-4-yl)-benzamide;
(+/−)-3-{1-[3-(1-Hydroxy-cyclohexyl)-propyl]-trans-3,4-dimethyl-piperidin-4-yl}-benzamide;
(+/−)-3-{1-[2-(4-Methoxy-phenyl)-ethyl]-trans-3,4-dimethyl-piperidin-4-yl}-benzamide;
(+/−)-3-{1-[2-(2-Methoxy-phenyl)-ethyl]-trans-3,4-dimethyl-piperidin-4-yl}-benzmide
(+/−)-3-{1-[2-(3-Methoxy-phenyl)-ethyl]-trans-3,4-dimethyl-piperidin-4-yl}benzamide
(+/−)-3-{trans-3,4-Dimethyl-1-[2-(3-trifluoromethyl-phenyl)-ethyl]-piperidin-4-yl}-benzamide;
(+/−)-3-{1-[2-(4-Cyano-phenyl)-ethyl]-trans-3,4-dimethyl-piperidin-4-yl}-benzamide;
(+)-3-{1-[3-(1-Hydroxy-cyclohexyl)-propyl]-trans-3,4-dimethyl-piperidin-4-yl}-benzamide
(−)-3-{1-[3-(1-Hydroxy-cyclohexyl)-propyl]-trans-3,4-dimethyl-piperidin-4-yl}-benzamide
(+/−)-3-{1-[2-(3-Bromo-phenyl)-ethyl]-trans-3,4-dimethyl-piperidin-4-yl}-benzamide
(+/−)-3-{1-[2-(4-Chloro-phenyl)-ethyl]-trans-3,4-dimethyl-piperidin-4-yl}-benzamide
(+/−)-3-{1-[2-(3-Chloro-phenyl)-ethyl]-trans-3,4-dimethyl-piperidin-4-yl}-benzamide
(+/−)-3-{1-[2-(3-Cyano-phenyl)-ethyl]-trans-3,4-dimethyl-piperidin-4-yl}-benzamide
(+/−)-3-{1-[2-(2,6-Dichloro-phenyl)-ethyl]-trans-3,4-dimethyl-piperidin-4-yl}-benzamide
(+/−)-3-[trans-3,4-Dimethyl-1-(2-pyridin-2-yl-ethyl)-piperidin-4-yl]-benzamide
(+/−)-3-[1-(2-Hydroxy-2-phenyl-ethyl)-trans-3,4-dimethyl-piperidin-4-yl]-benzamide
(+/−)-3-{1-[3-(1-Cyano-cyclohexyl)-propyl]-trans-3,4-dimethyl-piperidin-4-yl}-benzamide;
(+/−)-3-{1-[3-(1-Hydroxy-cyclopentyl)-propyl]-trans-3,4-dimethyl-piperidin-4-yl}-benzamide
(+/−)-3-{1-[3-(1-Methoxy-cyclohexyl)-propyl]-trans-3,4-dimethyl-piperidin-4-yl}-benzamide
(+/−)-3-{1-[3-(1-Hydroxymethyl-cyclopentyl)-propyl]-trans-3,4-dimethyl-piperidin-4-yl}-benzamide;
(+)-3-{1-[3-(1-Hydroxymethyl-cyclopentyl)-propyl]-trans-3,4-dimethyl-piperidin-4-yl}-benzamide
(−)-3-{1-[3-(1-Hydroxymethyl-cyclopentyl)-propyl]-trans-3,4-dimethyl-piperidin-4-yl}-benzamide;
(+)-3-[1-(2-Hydroxy-indan-2-ylmethyl)-trans-3,4-dimethyl-piperidin-4-yl]-benzamide
(+)-3-[1-(2-Hydroxy-indan-2-ylmethyl)-trans-3,4-dimethyl-piperidin-4-yl]-benzamide mesylate
(+)-(3-{1-[2-(2-Hydroxy-indan-2-yl)-ethyl]-trans-3,4-dimethyl-piperidin-4-yl}-benzamide
(+)-3-(1-{2-[3-(1-Hydroxy-cyclohexyl)-phenyl]-ethyl}-trans-3,4-dimethyl-piperidin-4-yl)-benzamide
(+)-3-[1-(cis-1-Hydroxy-3-phenyl-cyclobutylmethyl)-trans-3,4-dimethyl-piperidin-4-yl]-benzamide;
(+)-2-{2-[4-(3-Carbamoyl-phenyl)-trans-3,4-dimethyl-piperidin-1-yl]-ethyl}-indan-2-carboxylic acid amide;
(+)-3-{trans-3,4-Dimethyl-1-[3-(2-nitro-indan-2-yl)-propyl]-piperidin-4-yl}-benzamide
(+)-3-{1-[3-(2-Amino-indan-2-yl)-propyl]-trans-3,4-dimethyl-piperidin-4-yl}-benzamide
(+)-3-{1-[cis-3-(4-Bromo-phenyl)-1-hydroxy-cyclobutylmethyl]-trans-3,4-dimethyl-piperidin-4-yl}-benzamide
(+)-3-{1-[cis-1-Hydroxy-3-(4-methoxy-phenyl)-cyclobutylmethyl]-trans-3,4-dimethyl-piperidin-4-yl}-benzamide
(+)-3-{1-[2-(2-Amino-indan-2-yl)-ethyl]-trans-3,4-dimethyl-piperidin-4-yl}-benzamide
(+)-3-{1-[2-(2-Acetylamino-indan-2-yl)-ethyl]-trans-3,4-dimethyl-piperidin-4-yl}-benzamide; and
(+)-2-{2-[4-(3-Carbamoyl-phenyl)-trans-3,4-dimethyl-piperidin-1-yl]-ethyl}-indan-2-carboxylic acid.

The following compounds and their pharmaceutically acceptable salts are even more preferred:
(+/−)-3-(trans-3,4-Dimethyl-1-phenethyl-piperidin-4-yl)-benzamide;
(+/−)-3-(1-Indan-2-ylmethyl-trans-3,4-dimethyl-piperidin-4-yl)-benzamide;
(+/−)-3-(1-[3-(1-Hydroxy-cyclohexyl)-propyl]-trans-3,4-dimethyl-piperidin-4-yl)-benzamide;
(+/−)-3-{trans-3,4-Dimethyl-1-[2-(3-trifluoromethyl-phenyl)-ethyl]-piperidin-4-yl}-benzamide
(+/−)-3-{1-[2-(4-Cyano-phenyl)ethyl]-trans-3,4-dimethyl-piperidin-4-yl}-benzamide;
(+/−)-3-[1-(2-Hydroxy-2-phenyl-ethyl)-trans-3,4-dimethyl-piperidin-4-yl]-benzamide
(+/−)-3-{1-[3-(1-Hydroxymethyl-cyclopentyl)-propyl]-trans-3,4-dimethyl-piperidin-4-yl}-benzamide;
(+)-3-{1-[3-(1-Hydroxymethyl-cyclopentyl)-propyl]-trans-3,4-dimethyl-piperidin-4-yl}-benzamide;
(+)-3-{1-[3-(1-Hydroxy-cyclohexyl)-propyl]-trans-3,4-dimethyl-piperidin-4-yl}-benzamide;
(−)-3-{1-[3-(1-Hydroxy-cyclohexyl)-propyl]-trans-3,4-dimethyl-piperidin-4-yl}-benzamide;
(+)-3-[1-(2-Hydroxy-indan-2-ylmethyl)-trans-3,4-dimethyl-piperidin-4-yl]-benzamide
(+)-3-[1-(2-Hydroxy-indan-2-ylmethyl)-trans-3,4-dimethyl-piperidin-4-yl]-benzamide mesylate;
(+)-(3-{1-[2-(2-Hydroxy-indan-2-yl)-ethyl]-trans-3,4-dimethyl-piperidin-4-yl}-benzamide;
(+)-3-[1-(cis-1-Hydroxy-3-phenyl-cyclobutylmethyl)-trans-3,4-dimethyl-piperidin-4-yl]-benzamide; and
(+)-3-{1-[cis-1-Hydroxy-3-(4-methoxy-phenyl)-cyclobutylmethyl]-trans-3,4-dimethyl-piperidin-4-yl}-benzamide.

The present invention also relates to pharmaceutical compositions comprising any one or more of the above-described compounds in an effective amount, optionally in combination with a pharmaceutically acceptable carrier, excipient or additive.

The compounds of the present invention may be used to bind to and modulate (i.e., inhibit, activate or partially activate) an opioid receptor or receptors in a mammal, including a human. The present compounds exhibit pharmacological activity consistent with such binding. Compounds according to the present invention may also be used as reference materials, reference standards, including calibration standards and as synthetic intermediates.

The subject invention is also directed to pharmaceutical compositions comprising an effective amount of one or more compounds according to the invention as otherwise described herein, optionally in combination with a pharmaceutically acceptable additive, carrier or excipient.

The subject invention also provides a pharmaceutical composition for treating in a mammal, including a human, in need thereof a disease state, disorder or condition mediated by an opioid receptor or receptors which composition comprises an amount of a compound according to formula I or a pharmaceutically acceptable salt thereof effective in modulating an opioid receptor or receptors and a pharmaceutically acceptable carrier. In an embodiment of said pharmaceutical composition, the compound according to formula I is a compound of formula II or a pharmaceutically acceptable salt thereof.

The subject invention also provides a pharmaceutical composition for treating in a mammal, including a human, in need thereof a disorder or condition mediated by an opioid receptor or receptors which composition comprises an amount of a compound according to formula I or a pharmaceutically acceptable salt thereof effective in treating said disorder or condition and a pharmaceutically acceptable carrier. In an embodiment of said pharmaceutical composition, the compound according to formula I is a compound of formula II or a pharmaceutically acceptable salt thereof.

The subject invention also provides a pharmaceutical composition for treating in a mammal, including a human, in need thereof a disorder or condition selected from irritable bowel syndrome; constipation; nausea; vomiting; pruritic dermatoses, for example allergic dermatitis or contact dermatitis; psoriasis; eczema; an insect bite; an eating disorder, for example anorexia, bulimia, or obesity; depression, anxiety, schizophrenia; drug addiction, for example alcohol addiction, amphetamine addiction, cocaine addiction or addiction to an opioid, for example morphine, opium, or heroin; an opioid overdose; a sexual dysfunction, for example erectile dysfunction or impotence; stroke; head trauma; traumatic brain injury; spinal damage; Parkinson's disease; Alzheimer's disease, age-related cognitive decline; and Attention Deficit and Hyperactivity Disorder; which composition comprises an amount of a compound of formula I or a pharmaceutically acceptable salt thereof effective in modulating an opioid receptor or receptors and a pharmaceutically acceptable carrier. In an embodiment of said pharmaceutical composition, the compound according to formula I is a compound of formula II or a pharmaceutically acceptable salt thereof.

The subject invention also provides a pharmaceutical composition for treating in a mammal, including a human, in need thereof, a disorder or condition selected from irritable bowel syndrome; constipation; nausea; vomiting; pruritic dermatoses, for example allergic dermatitis or contact dermatitis; psoriasis; eczema; an insect bite; an eating disorder, for example anorexia, bulimia, or obesity; depression, anxiety, schizophrenia; drug addiction, for example alcohol addiction, amphetamine addiction, cocaine addiction or addiction to an opioid, for example morphine, opium, or heroin; an opioid overdose; a sexual dysfunction, for example erectile dysfunction or impotence; stroke; head trauma; traumatic brain injury; spinal damage; Parkinson's disease; Alzheimer's disease, age-related cognitive decline; and Attention Deficit and Hyperactivity Disorder; which composition comprises an amount of a compound of formula I or a pharmaceutically salt thereof effective in treating said disorder or condition and a pharmaceutically acceptable carrier. In an embodiment of said pharmaceutical composition, the compound according to formula I is a compound of formula II or a pharmaceutically acceptable salt thereof.

Another aspect of the subject invention is directed to treating in a mammal, including a human, in need thereof, a disorder or condition mediated by an opioid receptor or receptors which method comprises administering to said mammal an amount of a compound according to formula I, or a pharmaceutically acceptable salt thereof, effective in modulating an opioid receptor or receptors. In one embodiment of said method of treating a disorder or condition mediated by an opioid receptor or receptors, the compound according to formula I is a compound of formula II or a pharmaceutically acceptable salt thereof.

The subject invention also provides a method for treating in a mammal, including a human, in need thereof, a disease state, disorder or condition selected from irritable bowel syndrome; constipation; nausea; vomiting; pruritic dermatoses, for example allergic dermatitis or contact dermatitis; psoriasis; eczema; an insect bite; an eating disorder, for example anorexia, bulimia, and obesity; depression, anxiety, schizophrenia; drug addiction, for example alcohol addiction, amphetamine addiction, cocaine addiction or addiction to an opioid, for example morphine, opium, or heroin; an opioid overdose; a sexual dysfunction, for example erectile dysfunction or impotence; stroke; head trauma; traumatic brain injury; spinal damage; Parkinson's disease; Alzheimer's disease, age-related cognitive decline; and Attention Deficit and Hyperactivity Disorder; which method comprises administering to said mammal an amount of a compound of formula I or a pharmaceutically acceptable salt thereof as described, above effective to modulate an opioid receptor or receptors in said mammal. In one embodiment of said method, the compound according to formula I is a compound of formula II or a pharmaceutically acceptable salt thereof.

The subject invention also provides a method for treating in a mammal, including a human, in need thereof, a disease state, disorder or condition selected from irritable bowel syndrome; constipation; nausea; vomiting; pruritic dermatoses, for example allergic dermatitis or contact dermatitis; psoriasis; eczema; an insect bite; an eating disorder, for example anorexia, bulimia, or obesity; depression, anxiety, schizophrenia; drug addiction, for example alcohol addiction, amphetamine addiction, cocaine addiction and addiction to an opioid, for example morphine, opium, or heroin; an opioid overdose; a sexual dysfunction, for example erectile dysfunction or impotence; stroke; head trauma; traumatic brain injury; spinal damage; Parkinson's disease; Alzheimer's disease, age-related cognitive decline; and Attention Deficit and Hyperactivity Disorder; which method comprises administering to said mammal an amount of a compound of formula I or a pharmaceutically acceptable salt thereof as described above effective in treating said disease state, disorder or condition in said mammal. In one embodiment of said method, the compound according to formula I is a compound of formula II or a pharmaceutically acceptable salt thereof.

Thus, compounds of the present invention are useful because they possess pharmacological activity in animals, especially mammals, including humans. These compounds may also find use as standards in analytical assays or as intermediates in the synthesis of final compounds exhibiting pharmacological activity.

The subject invention also provides a method for treating in a mammal, including a human, in need thereof a disorder or condition mediated by an opioid receptor or receptors which method comprises administering to said mammal an amount of a compound according to formula I effective in treating said disorder or condition. In one embodiment of said method, the compound according to formula I is a compound of formula II or a pharmaceutically acceptable salt thereof.

Methods of synthesizing compounds according to the present invention and key intermediates which can be in such methods are additional aspects of the present invention. These methods are described in greater detail herein below.

The present invention also relates to methods of synthesizing compounds according to the present invention as described in greater detail herein.

### DETAILED DESCRIPTION OF THE INVENTION

The following terms shall be used to describe the subject invention.

The term “compound”, as used herein, unless otherwise indicated, refers to any specific chemical compound disclosed herein. Within its use in context, the term generally refers to a single compound, but in certain instances may also refer to stereoisomers and/or optical isomers (including racemic mixtures), as well as specific enantiomers or enantiomerically enriched mixtures of disclosed compounds.

The term “effective” is used herein, unless otherwise indicated, to describe an amount of a compound which, in context, is used to produce or effect an intended result, whether that result relates to the treatment of a disease state, disorder or condition or alternatively, is used to produce another compound, agent or composition.

The terms “treatment”, “treating”, and the like, refers to reversing, alleviating, or inhibiting the progress of the disorder or condition to which such term applies, or one or more symptoms of such disorder or condition. As used herein, these terms also encompass, depending on the condition of the patient, preventing the onset of a disorder or condition, or of symptoms associated with a disorder or condition, including reducing the severity of a disorder or condition or symptoms associated therewith prior to affliction with said disorder or condition. Thus, “treatment”, as used herein, can refer to administration of a compound of the invention to a subject that is not at the time of administration afflicted with the disorder or condition. “Treating” thus also encompasses preventing the recurrence of a disorder or condition or of symptoms associated therewith.

The term “addiction”, as used herein, for example in “drug addiction”, including “alcohol addiction”, unless otherwise indicated, refers to a maladaptive use of a substance, which may be either with physiological dependence or without. The term “addiction” thus includes both substance abuse (e.g. alcohol, amphetamine, cocaine or an opioid, for example morphine, opium, or heroin abuse) and substance dependence (e.g. alcohol, amphetamine, cocaine or an opioid, for example morphine, opium, or heroin dependence). The maladaptive pattern of substance use may manifest itself in recurrent and significant adverse consequences related to the repeated use of the substance. The recurrent substance use may result in a failure to fulfill major role obligations at work, school, or home. The maladaptive use of a substance may involve continued use of the substance despite persistent or recurrent social or interpersonal problems caused or exacerbated by the effects of the substance (e.g., arguments with spouse, physical fights). The maladaptive pattern of substance use may involve clinically significant impairment or distress, for example manifested by tolerance for the substance, withdrawal symptoms, self-injurious behavior, unsuccessful efforts to cut down or control the substance use, and/or taking larger amounts of the substance and/or taking amounts of the substance over a longer period than was intended. Substances to which an addiction may be formed include, but are not limited to, the drugs recited above, as well as others, for example benzodiazepines such as Valium®.

The term ‘mammal’, as used herein, and unless otherwise indicated, means any mammal. The term ‘mammal’ includes, for example and without limitation, dogs, cats, and humans. The term “patient” or “subject” may be alternatively used to describe such a mammal, including a human, to whom treatment or use with the compounds or compositions according to the subject invention is provided. For treatment or use with/or of those disease states, conditions or disease states which are specific for a specific animal (especially, for example, a human subject or patient), the term patient or subject refers to that particular animal.

References herein to disease states, disorders and conditions “mediated by an opioid receptor or receptors” indicate disorders or conditions the treatment of which can be facilitated by modulating (i.e. inhibiting, partially inhibiting, activating, or partially activating) an opioid receptor or receptors. Examples of disorders and conditions the treatment of which is facilitated by modulation of an opioid receptor or receptors include, but are not limited to; irritable bowel syndrome, eating disorders, sexual dysfunction, depression, anxiety, schizophrenia and drug addictions, as well as the other specific disorders and conditions recited herein:

The term “alkyl”, as used herein, unless otherwise indicated, includes saturated monovalent hydrocarbon radicals having straight, cyclic or branched moieties. Examples of alkyl groups include, but are not limited to, methyl, ethyl, propyl, isopropyl, sec-butyl and t-butyl. Within context, the use of the term “alkyl” may also subsume the use of or refer to alkylene groups, i.e., a hydrocarbon radical derived from alkyl groups which are diradicals, rather than monoradicals.

The term “cycloalkyl”, as used herein, unless otherwise indicated, includes non-aromatic saturated cyclic alkyl moieties wherein alkyl is as defined above. Examples of cycloalkyl include, but are not limited to, cyclopropyl, cyclobutyl, cyclopentyl, cyclohexyl, and cycloheptyl.

The term “carbocyclic”, as used herein, unless otherwise indicated, refers to a cyclic group in which all of the atoms of the ring are carbon atoms. Representative carbocyclic groups include cycloalkyl groups as described above. The term carbocyclic subsumes the term aryl within it.

The term “heterocyclic”, as used herein, unless otherwise indicated, refers to a cyclic group in which at least one atom of the ring is a heteroatom (i.e., O, S or N). The term heterocyclic subsumes the terms heteroaryl and heterocycloalkyl within it. Thus, a 5- to 7-membered heterocyclic group subsumes a 5- to 7-membered heteroaryl group within it.

The term “aryl”, as used herein, unless otherwise indicated, includes an organic radical derived from an aromatic hydrocarbon by removal of one hydrogen, such as phenyl, naphthyl, indenyl, and fluorenyl.

The term “heteroaryl”, as used herein, refers to aromatic groups containing one or more heteroatoms (O, S, or N), preferably from one to four heteroatoms. A multicyclic group containing one or more heteroatoms wherein at least one ring of the group is aromatic is a “heteroaryl” group. The heteroaryl groups of this invention can also include ring systems substituted with one or more oxo moieties. Examples of heteroaryl groups are pyridinyl, pyridazinyl, imidazolyl, pyrimidinyl, pyrazolyl, triazolyl, pyrazinyl, quinolyl, isoquinolyl, tetrazolyl, furyl, thienyl, isoxazolyl, thiazolyl, oxazolyl, isothiazolyl, pyrrolyl, indolyl, benzimidazolyl, benzofuranyl, cinnolinyl, indazolyl, indolizinyl, phthalazinyl, triazinyl, isoindolyl, purinyl, oxadiazolyl, thiadiazolyl, furazanyl, benzofurazanyl, benzothiophenyl, benzotriazolyl, benzothiazolyl, benzoxazolyl, quinazolinyl, quinoxalinyl, naphthyridinyl, dihydroquinolyl, tetrahydroquinolyl, dihydroisoquinolyl, tetrahydroisoquinolyl, benzofuryl, furopyridinyl, pyrolopyrimidinyl, and azaindolyl. The foregoing groups, as derived from the compounds listed above, may be C-attached or N-attached where such is possible. For instance, a group derived from pyrrole may be pyrrol-1-yl (N-attached), pyrrol-2-yl or pyrrol-3-yl (C-attached). The terms referring to the groups also encompass all possible tautomers.

The term “reductive amination”, as used herein, refers to any process whereby the combination of an aldehyde or a ketone, or aldehyde or ketone equivalent, such as a bisulfite addition complex of an aldehyde, is combined with, in reference to the subject invention, a primary amine, secondary amine or ammonia, or ammonia source, such that the compounds condense to generate an intermediate imine or iminium ion that may be subjected to reduction by means of hydrogenation, such as mediated by a metal species such as palladium or platinum in many forms useful for reduction and a hydrogen source, such as hydrogen gas, or any precursor to hydrogen gas, including but not limited to formate derivatives or cyclohexadiene, or other hydride sources whereby hydride delivery from said source occurs by mechanisms commonly understood and employed. These include hydride reagents such as boron or aluminum hydride sources, for instance borohydrides, such as [(X)ₙBH₄₋ₙ]⁻ (n=0, 1, 2, 3) or aluminum hydrides such as [(X)AlH₄₋ₙ]⁻ (n=0, 1, 2, 3) (wherein X may be any of the commonly cited ligands for transformations such a reductive amination including but not limited to acetoxy, trifluoroacetoxy, alkoxy, or lower alkyl for boron or alkoxy or lower alkyl for aluminum). Other hydrides may be equally suited to these transformations (for instance silanes or stannanes).

The term “reducing” or “reductive conditions”, as used herein, refers to any process whereby dehydrohalogenation, hydrogenolysis, hydrogenation, or reduction of unsatured bonds occurs as desired.

The term “leaving group” or “alkylating agent”, as used herein, refers to any group or agent suitable in the conversion of a primary amine, secondary amine or ammonia or ammonia source that effectively departs in a bond-forming event from a carbon atom of interest, such as in an alkylation reaction. Suitable groups or agents include halides (iodide, bromide or chloride), sulfonates (such methane sulfonate, trifluoromethanesulfonate or, aryl sulfonates such as tosyl or nosyl groups), epoxides or aziridines or any variation that is well known to those of skill in the art. In addition, the processes involving leaving groups or alkylating agents may be employed in the formation of other C—X bonds where the nucleophile X is oxygen, sulfur, or carbon centered.

Pharmaceutical salts of compounds according to the present invention are an important aspect. Pharmaceutical salts of compounds of formula I or II can be obtained by forming salts with any acidic or basic group present on a compound of formula I or II. Examples of pharmaceutically acceptable salts of the compounds of formula I are the salts of hydrochloric acid, p-toluenesulfonic acid, fumaric acid, citric acid, succinic acid, salicylic acid, oxalic acid, hydrobromic acid, phosphoric acid, methanesulfonic acid, tartaric acid, maleic acid, di-p-toluoyl tartaric acid, acetic acid, sulfuric acid, hydroiodic acid, mandelic acid, sodium, potassium, magnesium, calcium, and lithium. Mesylate and/or citrate salts may be particularly preferred in the subject invention.

As noted above, the compounds of formula I may have optical centers and therefore may occur in different enantiomeric and other stereoisomeric configurations. The invention includes all enantiomers, diastereomers, and other stereoisomers of such compounds of formula I, as well as racemic and other mixtures thereof.

Pharmaceutically acceptable salts of a compound of formula I can be prepared in a conventional manner by treating a solution or suspension of the corresponding free base or acid with one chemical equivalent of a pharmaceutically acceptable acid or base. Conventional concentration or crystallization techniques can be employed to isolate the salts. Illustrative of suitable acids are acetic, lactic, succinic, maleic, tartaric, citric, gluconic, ascorbic, benzoic, cinnamic, fumaric, sulfuric, phosphoric, hydrochloric, hydrobromic, hydroiodic, sulfamic, sulfonic acids such as methanesulfonic, benzene sulfonic, p-toluenesulfonic, and related acids. Mesylate and/or citrate salts may be preferred in the subject invention. Illustrative bases are sodium, potassium, and calcium.

A compound of this invention may be administered alone or in combination with pharmaceutically acceptable carriers, in either single or multiple doses. Suitable pharmaceutical carriers include inert solid diluents or fillers, sterile aqueous solutions and various organic solvents. The pharmaceutical compositions formed by combining a compound of formula I or a pharmaceutically acceptable salt thereof can then be readily administered in a variety of dosage forms such as tablets, powders, lozenges, syrups, injectable solutions and the like. These pharmaceutical compositions can, if desired, contain additional ingredients such as flavorings, binders, excipients and the like. Thus, for purposes of oral administration, tablets containing various excipients such as sodium citrate, calcium carbonate and calcium phosphate may be employed along with various disintegrants such as starch, methylcellulose, alginic acid and certain complex silicates, together with binding agents such as polyvinylpyrrolidone, sucrose, gelatin and acacia. Additionally, lubricating agents such as magnesium stearate, sodium lauryl sulfate and talc are often useful for tabletting purposes. Solid compositions of a similar type may also be employed as fillers in soft and hard filled gelatin capsules. Preferred materials for this include lactose or milk sugar and high molecular weight polyethylene glycols. When aqueous suspensions or elixirs are desired for oral administration, the essential active ingredient therein may be combined with various sweetening or flavoring agents, coloring matter or dyes and, if desired, emulsifying or suspending agents, together with diluents such as water, ethanol, propylene glycol, glycerin and combinations thereof.

For parenteral administration, solutions containing a compound of this invention or a pharmaceutically acceptable salt thereof in sesame or peanut oil, aqueous propylene glycol, or in sterile aqueous solution may be employed. Such aqueous solutions should be suitably buffered if necessary and the liquid diluent first rendered isotonic with sufficient saline or glucose. These particular aqueous solutions are especially suitable for intravenous, intramuscular, subcutaneous and intraperitoneal administration. The sterile aqueous media employed are all readily available by standard techniques known to those skilled in the art.

A compound of formula I or a pharmaceutically acceptable salt thereof can be administered orally, transdermally (e.g., through the use of a patch), parenterally (e.g. intravenously), rectally, topically, or by inhalation. In general, the daily dosage for treating a disorder or condition as described herein using a compound of formula I will be about from about 0.01 to about 100 mg per kg, preferably from about 0.1 to about 10 mg per kg, of the body weight of the animal to be treated. As an example, a compound of the formula I, or a pharmaceutically acceptable salt thereof, can be administered for treatment to an adult human of average weight (about 70 kg) in a dose ranging from about 0.5 mg up to about 10 g per day, preferably from about 10 mg to about 1 g per day, in single or divided (i.e., multiple) portions. Variations based on the aforementioned dosage ranges may be made by a physician of ordinary skill taking into account known considerations such as the weight, age, and condition of the animal being treated, the severity of the affliction, and the particular route of administration chosen.

Compounds of formula I of the present invention have been found to display activity in opioid receptor binding assays selective for the mu, kappa and delta opioid receptors. Assays for mu, kappa and delta opioid receptor binding can be performed according to the following procedure:

Affinity of a compound for the delta opioid receptor can be assessed using binding of the delta opioid receptor ligand [³H]-naltrindole to NG108-15 neuroblastoma-glioma cells according to modification of the protocol described in Law et al. (Law, P. Y., Koehler, J. E. and Loh, H. H., “Comparison of Opioid Inhibition of Adenylate Cyclase Activity in Neuroblastoma N18TG2 and Neuroblastoma X Glioma NG108-15 Hybrid Cell Lines”. Molecular Pharmacology, 21: 483-491 (1982)). Law et al. is incorporated herein in its entirety by reference. Affinity of a compound for the kappa opioid receptor can be assessed using binding of [³H]-bremazocine to kappa receptors as described in Robson, L. E., et al., “Opioid Binding Sites of the Kappa-type in Guinea-pig Cerebellum”, Neuroscience (Oxford), 12(2): 621-627 (1984). Robson et al. is incorporated herein it its entirey by reference. For assessment of a compound for mu opioid receptor activity, the mu receptor ligand [3H]-DAMGO (Perkin Elmer Life Sciences, Boston, Mass.; specific activity 55 Ci/mmol, 1.5 nM) is used with rat forebrain tissue. Briefly, the binding is initiated with the addition of a crude membrane preparation of rat forebrain tissue to 96-well polypropylene plates containing the radioligand [³H]-DAMGO and test compound, and are incubated for about 90 minutes at about 25° C. The assay is terminated by rapid filtration with 50 mM Tris HCl pH 7.4 onto Wallac Filtermat B and counted on a Betaplate reader (Wallac).

The data generated can be analyzed using IC₅₀ analysis software in Graphpad Prism. Ki values can be calculated using Graphpad Prism according to the following formula:
Ki=IC₅₀/1+[³H ligand]/K_{D}
where IC₅₀ is the concentration at which 50% of the ³H ligand is displaced by the test compound and K_{D} is the dissociation constant for the ³H ligand at the receptor site.

### Biological Activity

Other assays which may be used for determining the binding of compounds according to the present invention to opioid receptors are well known in the art. These assays may be used to assess the ability of a compound to modulate (i.e., inhibit, partially inhibit, activate or partially activate) an opioid receptor or receptors by determining a compound's agonist or antagonist activity in the in vitro or in vivo assay. These assays include, for example, the GTP gamma S binding assay as described in Martin, et al., J. Pharm. Exp. Ther., 301, 661-671 (2003) and Zaki, et al., J. Pharm. Exp. Ther., 298, 1015-1020 (2002), as well as other binding assays, such as the isolated guinea pig ileum and receptor binding assay as disclosed, for example, by Takayama, et al., J. Med. Chem., 45, 1949-1956 (2002) and the guinea pig brain binding assay as described by Wentland, et al., J. Med. Chem., 46, 838-849 (2003). The use of mouse brain tissue to determine the functional activity of the compounds of interest is another binding assay which can be used for characterizing the modulation of the present compounds at opioid receptors, as disclosed by Martin, et al., Idem. Other binding assays include the tail-flick assay in mice or the radiant heat paw-withdrawal hyperalgesic testing in mice, as described by Hosohata, et al., J. Pharm. Exp. Ther., 304, 683-688 (2003), among others. These assays or variations of these assays are well-known to those of ordinary skill in the art.

The Ki values of the specific compounds of formula I of Examples 1-4, infra, in a mu opioid receptor-binding assay to brain tissue such as that described above were determined. These compounds were all found to have Ki values of about 200 nM or less for the mu receptor. The compounds of formula I are biologically advantageous in that they are not metabolized by the p450 isozyme CYP2D6 to an extent which could possibly cause significant dosing or pharmacokinetic variations. Since variability in the presence of CYP2D6 in the human population exists, it is beneficial to have a medicine that is not metabolized by CYP2D6 because effective dosages across the human population will be independent of CYP2D6 differences.

Whether a compound is metabolized by CYP2D6 can be determined using CYP2D6, for example that purchased from PanVera Corporation (Madison, Wis.). Identification of compounds that are subsrates of CYP2D can be determined, for example, according to the following assay. Compounds are incubated with human recombinant CYP2D6′ BACULOSOMES™ (PanVera Corporation; Madison, Wis.). More particularly, compound (1 uM), rCYP2D6 (2.8 pmol/ml), buffer (100 mM phosphate, pH=7.4) and NADPH (1.67 mg/ml, Sigma Aldrich #201-210) are incubated at 37° C. Aliquots (50 ul) are taken at 0, 5, 10, 20 and 30 minutes, and the reaction is quenched by addition of ice cold sodium carbonate buffer (50 ul, 20 mM pH=10.5, with internal standard). The resulting solution is extracted (10× volume of tert-butyl methyl ester) and samples were analyzed by LC/MS. Loss of parent compound is monitored, and half-life for parent compound disappearance is calculated using WinNonlin.

### Synthetic Methods

The synthetic methods described below in the “Detailed Description” section and in the following Examples produce primarily compounds of formula I having the relative stereochemistry illustrated by compounds of formula II depicted below: 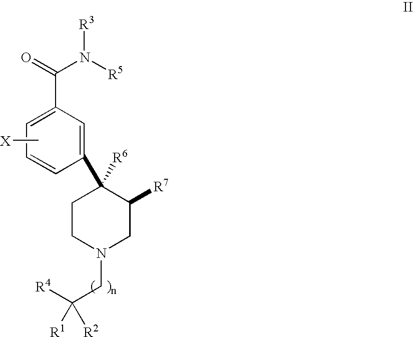
Compounds of formula II, and their pharmaceutically acceptable salts, are preferred embodiments of the invention.

The subject invention also includes isotopically-labeled compounds; which are identical to those recited in formula I, but for the fact that one or more atoms are replaced by an atom having an atomic mass or mass number different from the atomic mass or mass number usually found in nature. Examples of isotopes that can be incorporated into compounds of the invention include isotopes of hydrogen, carbon, nitrogen, oxygen, phosphorous, fluorine, iodine, and chlorine, such as ³H, ¹C, ¹⁴C, ¹⁶F, 1231 and ¹²⁵I. Compounds of the present invention and pharmaceutically acceptable salts of said compounds that contain the aforementioned isotopes and/or other isotopes of other atoms are within the scope of this invention. Isotopically-labeled compounds of the present invention, for example those into which radioactive isotopes such as ³H and ¹⁴C are incorporated, are useful in drug and/or substrate tissue distribution assays. Tritiated, i.e., ³H, and carbon-14, i.e., ¹⁴C, isotopes are particularly preferred for their ease of preparation and detectability. ¹¹C and ¹⁸F isotopes are particularly useful in PET (positron emission tomography), and ¹²⁵I isotopes are particularly useful in SPECT (single photon emission computerized tomography), all useful in brain imaging. Further, substitution with heavier isotopes such as deuterium, i.e., ²H, can afford certain therapeutic advantages resulting from greater metabolic stability, for example increased in vivo half-life or reduced dosage requirements and, hence, may be preferred in some circumstances. Isotopically labeled compounds of formula I of this invention can generally be prepared by carrying out the procedures disclosed in the Schemes and/or in the Examples below, by substituting a readily available isotopically labeled reagent for a non-isotopically labeled reagent.

Accordingly, the subject invention also provides a compound of formula I wherein one or more atoms thereof have an atomic mass or mass number different from the atomic mass or mass number usually found in nature, or a pharmaceutically acceptable salt of such compound. The subject invention also provides a method for obtaining an image of opioid receptors in a mammalian, including a human, subject which method comprises administering to said subject an amount of an isotopically-labeled compound of formula I, or pharmaceutically acceptable salt thereof, effective in imaging opioid receptors in said subject.

Pharmaceutical salts of the above-described compounds are another important aspect. Pharmaceutical salts of compounds of formula I can be obtained by forming salts with any acidic or basic group present on a compound of formula I. Examples of pharmaceutically acceptable salts of the compounds of formula I are the salts of hydrochloric acid, p-toluenesulfonic acid, fumaric acid, citric acid, succinic acid, salicylic acid, oxalic acid, hydrobromic acid, phosphoric acid, methanesulfonic acid, tartaric acid, maleic acid, di-p-toluoyl tartaric acid, acetic acid, sulfuric acid, hydroiodic acid, mandelic acid, sodium, potassium, magnesium, calcium, and lithium, among others. Mesylate and/or citrate salts may be particularly preferred in the subject invention.

Compounds of the formula I, above, and their pharmaceutically acceptable salts, can be prepared according to the following reaction Schemes I through IV as discussed. Unless otherwise indicated X, n and R¹ through R⁷ are as defined above. Isolation and purification of the products is accomplished by standard procedures, which are known to a chemist of ordinary skill.

As used herein, the expression “reaction inert solvent” refers to a solvent system in which the components do not interact with starting materials, reagents, or intermediates of products in a manner which adversely affects the yield of the desired product.

During any of the following synthetic sequences it may be necessary and/or desirable to protect sensitive or reactive groups on any of the molecules concerned. This may be achieved by means of conventional protecting groups, such as those described in T. W. Greene, Protective Groups in Organic Chemistry, John Wiley & Sons, 1981; and T. W. Greene and P. G. M. Wuts, Protective Groups in Organic Chemistry, 3^{rd} Edition, John Wiley & Sons, 1999.

Scheme I illustrates a method for the preparation of compounds having the basic structure of formula I, where X═H, R⁶ and R⁷=methyl, R³ and R⁵═H and R¹, R² and R⁴ are defined as above.

Referring to Scheme I, a bis-meslyate of formula (II) can be treated with N-benzylamine and sodium carbonate as described in WO 9959971, which provided the desired amine of formula (III). Treatment of a compound of formula (III) with zinc cyanide, in the presence of a suitable catalyst, such as tetrakistriphenylphosphine palladium (0), in solvents such as dimethylformamide, at temperatures ranging from room temperature to about the reflux temperature, preferably at about 85° C., produces the corresponding nitrile of formula (IV). Oxidation of a nitrile of formula (IV) with dilute hydrogen peroxide, in the presence of a suitable alkali metal base, such as sodium carbonate, in solvents such as dimethylformamide or dimethylsulfoxide, at temperatures ranging from 0° C. to about room temperature, preferably at about room temperature, produces the corresponding amide of formula (V). Finally, compounds of formula (VI) can be prepared by treatment of compounds of formula (V) with hydrogen gas (at pressures ranging from atmospheric to 50 psi) in the presence of a suitable catalyst such as palladium on carbon, in alcoholic solvents such as methanol, at temperatures ranging from room temperature to 60° C., preferably at about room temperature, which produces the compound of formula (VI). 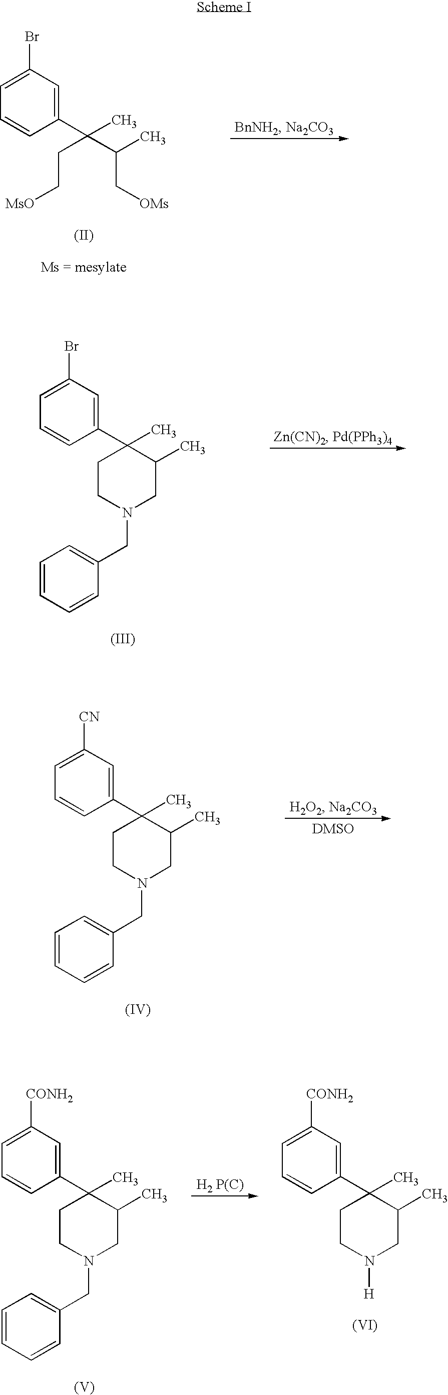
Referring to Scheme II, treatment of a compound of general formula (VII) with an appropriately substituted aldehyde of formula (VIII) and a reducing agent such as sodium triacetoxyborohydride, in the presence of acetic acid, in solvents such as dichloromethane or dichloroethane, at temperatures ranging from 0° C. to about room temperature, preferably at about room temperature, produce the corresponding compounds of formula (IX). Alternatively, compounds of formula (IX) can also be prepared by treatment of a compound of formula (VII) with a suitable alkylating reagent of formula (X). This reaction should be carried out in the presence of a suitable base, such as potassium carbonate, in solvents such as acetonitrile, at temperatures ranging from room temperature to about the reflux temperature, preferably at about the reflux temperature, which produces the desired compounds of formula (IX). Reagents (VII) and (X) can be prepared using methods that are known to one of ordinary skill in the art. 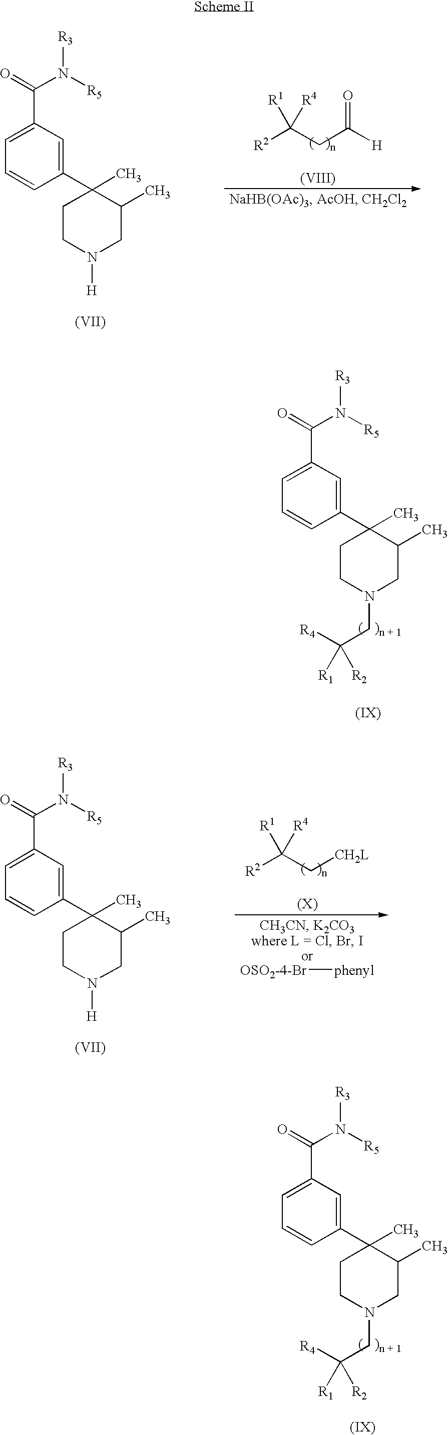
Referring to Scheme III below, compounds of formula (IXa) can be prepared by treatment of a compound of formula (VII) with a reagent of formula (XI) wherein R^{18a} is oxygen or —NH, NR, NHSO₂R or NCOR. This reaction should be carried out in the presence of a suitable base such as triethyl amine, in alcoholic solvents such as ethanol, at temperatures ranging from room temperature to about the reflux temperature, preferably at about the reflux temperature to produce the desired compound of formula (IX). Alternatively, compounds of formula (IX) can also be prepared by treatment of a compound of formula (VII) with an appropriately substituted acid chloride of formula (XII). The reaction should be carried out in the presence of a suitable base such as Et₃N or pyridine, in solvents such as tetrahydrofuran or methylene chloride, at temperature ranging from 0° C. to room temperature, preferably at about room temperature. The amide products from this reaction (not depicted) are then reduced with a suitable reducing agent such as lithium aluminum hydride, in solvents such as ethyl ether or, tetrahydrofuran, at temperatures ranging from room temperature to about the reflux temperature, preferably at about the reflux temperature, which produce the desired products of formula (IX). Reagents (XI) and (XII) can be prepared using methods that are known to one of ordinary skill in the art. 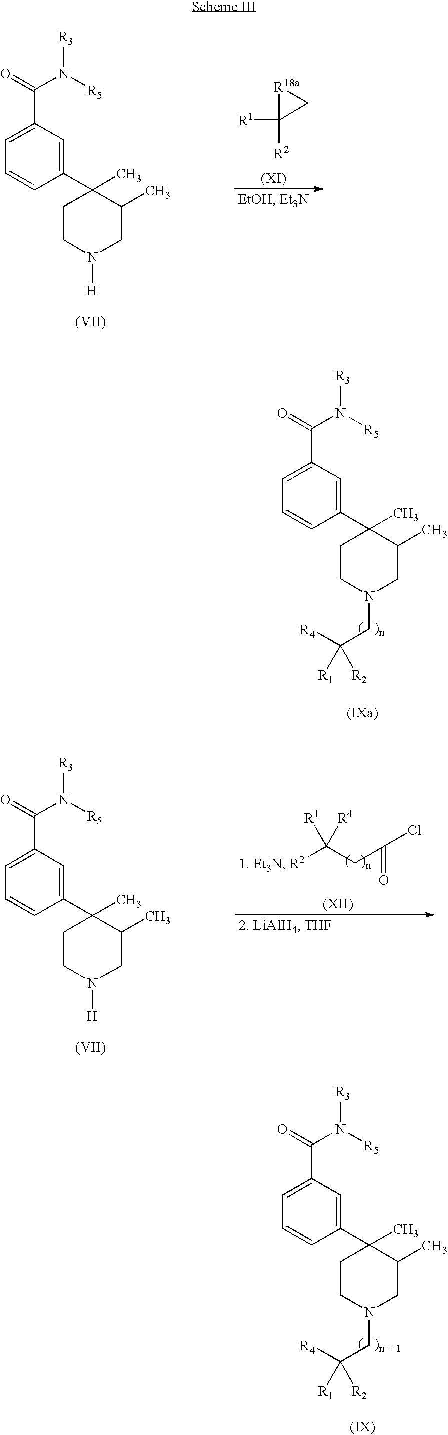
Scheme IV below further illustrates a method for the preparation of compounds having the basic structure of formula I, where X═H, R⁶ and R⁷=methyl, R¹, R², R³, R⁴ and R⁵ are described as above. Referring to scheme IV below, a compound of formula (XIII) (the preparation of which is described in patent publication EP 1072592 as a source for the starting material), is placed under a carbon monoxide atmosphere at a pressure ranging from about 14 to 100 psi, in a solution of dimethylsulfoxide and a lower alkanol such as methanol or ethanol, with a suitable trialkylamine base (e.g., triethylamine) and palladium acetate with 1,3-bis(diphenylphosphino)propane (DPPP) or another suitable palladium ligand. Other suitable palladium catalysts such as bis(triphenylphosphine)palladium dichloride may also be used. This reaction is performed at temperatures ranging from about 20° C. to 100° C. to produce the corresponding ester of formula (XIV). Compounds of formula (XV) can be prepared by treatment of compounds of formula (XIV) with hydrogen gas (at pressures ranging from atmospheric to 50 psi) in the presence of a suitable catalyst such as palladium on carbon, in alcoholic solvents such as methanol, at temperatures ranging from room temperature to 60° C., preferably at about room temperature, to produce the corresponding secondary amine of formula (XV). Treatment (XV) with an appropriately substituted aldehyde of formula (VIII) and a reducing agent such as sodium triacetoxyborohydride, in the presence of acetic acid, in solvents such as dichloromethane or dichloroethane, at temperatures ranging from 0° C. to about room temperature, preferably at about room temperature, produce the corresponding compound of formula (XVI). Finally, treatment of the ester of formula (XVI) with an aluminum amide of a primary or secondary amine, for example, methylamine, in a solvent such as dichloroethane or toluene, at a temperature ranging from about 20° C. to about the reflux temperature, preferably at about the reflux temperature, yields the corresponding amide compounds of formula (IX). 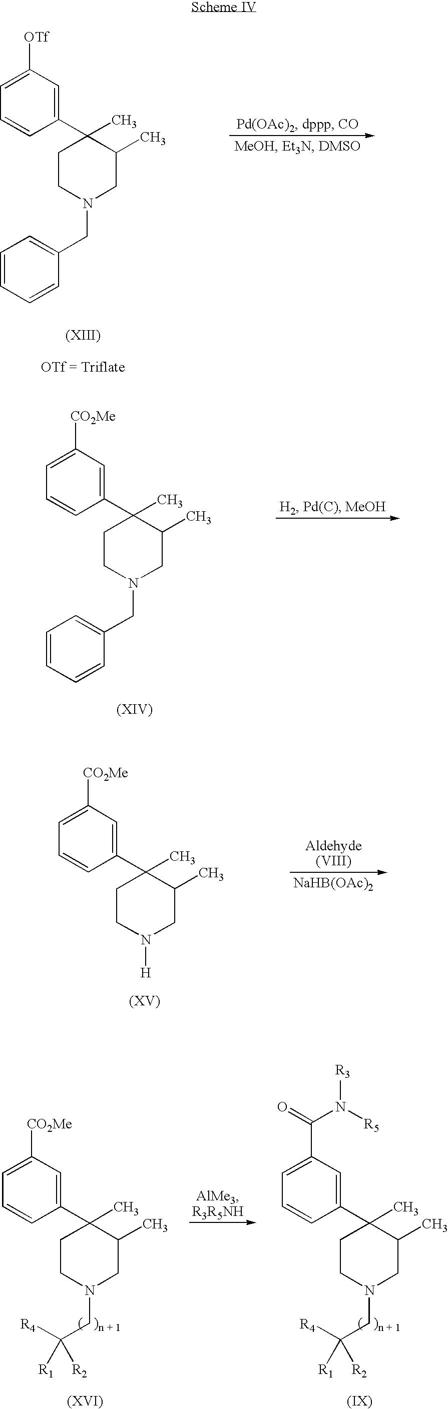
The following Examples illustrate the present invention. It is to be understood, however, that the invention, as fully described herein and as recited in the claims, is not intended to be limited by the details of the following examples.

### EXAMPLES

### Preparation 1

### (+)-1-Benzyl-4-(3-bromo-phenyl)-trans-3,4-dimethyl-piperidine

To a solution of (+)-3-(3-Bromophenyl)-trans-2,3-dimethyl-5-((methansulfonyl)-oxy)pentyl methanesulfonate [WO 9959971] (18.8 g, 42.5 mmol) in 75 mL of anhydrous toluene was added sodium carbonate (9.46 g, 89.2 mmol) in 40 mL water, followed by benzyl amine (11.6 mL, 106.2 mmol). The reaction mixture was heated to 105° C. for 24 hours. Another portion of benzyl amine (2.32 mL, 21.2 mmol) was added and the mixture was heated for a further 24 hours. The mixture was cooled to room temperature, treated with 25 mL of water, heated to 40° C. and treated with succinic anhydride (1.7 equivalents). The biphasic mixture was cooled to room temperature, the layers were separated and the aqueous layer was extracted with toluene. The combined organic layers were washed with water, brine solution and dried over anhydrous MgSO₄. Filtration and concentration of the resulting solution afforded the desired product (15.1 g) as a pale yellow oil. 400 MHz ¹H NMR (CDCl₃) δ 7.14-7.39 (m, 9H), 3.51 (ABq, ΔAB=53.6 Hz, J=13 Hz, 2H), 2.81-2.83 (m, 1H), 2.52 (brs, 2H), 2.30-2.38 (m, 2H), 1.94 (brs, 1H), 1.55-1.57 (m, 1H), 1.29 (s, 3H), 0.77 (d, J=7.05 Hz, 3H).

### Preparation 2

### (+)-3-(1-Benzyl-trans-3,4-dimethyl-piperidin-4-yl)-benzonitrile

To a stirring solution of (+)-1-Benzyl-4-(3-bromo-phenyl)-trans-3,4-dimethyl-piperidine prepared as described above (2.0 g, 5.58 mmol) in 30 mL anhydrous DMF at room temperature was added zinc cyanide (983 mg, 8.37 mmol) and tetrakistriphenylphosphine palladium (0) (3.22 g, 2.79 mmol). The mixture was cooled to −78° C. and de-oxygenated with vacuum/N₂ purge. The mixture was warmed to room temperature and then heated at 85° C. for 3 hours. The mixture was cooled to room temperature and diluted with ethyl acetate and water. The layers were separated, the aqueous layer extracted with ethyl acetate, the combined organic layers were dried over MgSO₄ and filtered through a small silica gel plug. The solution was concentrated to yield an amber oil which was purified by flash chromatography using 40% Ethyl Acetate/Hexanes. The product containing fractions were collected and concentrated to give 1.2 gm (70%) of the desired product. 400 MHz ¹H NMR (CDCl₃) δ 7.20-7.54 (m, 9H), 3.50 (ABq, ΔAB=56.0 Hz, J=13.3 Hz, 2H), 2.83-2.86 (m, 1H), 2.44-2.51 (m, 2H), 2.27-2.39 (m, 2H), 1.95-1.97 (m, 1H), 1.56-1.59 (m, 1H), 1.29 (s, 3H), 0.72 (d, J=7.05 Hz, 3H); MS (M+1)=305.2.

### Preparation 3

### (+)-3-(1-Benzyl-trans-3,4-dimethyl-piperidin-4-yl)-benzamide

To a stirring solution of (+)-3-(1-Benzyl-trans-3,4-dimethyl-piperidin-4-yl)-benzonitrile prepared above (1.92 g, 6.30 mmol) in 60 mL DMSO at room temperature was added 30% H₂O₂ (3.22 mL, 31.5 mmol) and potassium carbonate (122 mg, 0.88 mmol). After stirring 18 hours, the mixture was diluted with water and extracted with ethyl acetate. The combined organic layers were dried over MgSO₄ and concentrated to yield 1.95 g of product. 400 MHz ¹H NMR (CDCl₃) δ 7.76-7.77 (m, 1H), 7.54-7.55 (m, 1H), 7.52-7.54 (m, 1H), 7.19-7.44 (m, 6H), 6.15 (brs, 2H), 3.50 (ABq, ΔAB=56.5 Hz, J=13.3 Hz, 2H), 2.82-2.84 (m, 1H), 2.52-2.56 (m, 2H), 2.37-2.42 (m, 2H), 21.99-2.01 (m, 1H), 1.60-1.63 (m, 1H), 1.30 (s, 3H), 0.73 (d, J=7.05 Hz, 3H); MS (M+1)=323.2.

### Preparation 4

### (+)-3-(trans-3,4-Dimethyl-piperidin-4-yl)-benzamide

In a 500 mL Parr bottle, (+)-3-(1-Benzyl-trans-3,4-dimethyl-piperidin-4-yl)-benzamide (prepared as described above, 1.94 g, 6.02 mmol) was dissolved in 100 mL methanol at room temperature. To this solution was added 500 mg of 10% Pd(C). The mixture was hydrogenated under 50 psi H₂ at 60° C. for 18 hours. The mixture was cooled to room temperature and filtered through a plug of celite and the pad was washed several times with methanol. The resulting solution was concentrated under reduced pressure to yield 1.32 gm of desired product. 400 MHz ¹H NMR (CD₃OD) δ 7.80-7.81 (m, 1H), 7.65-7.66 (m, 1H), 7.47-7.49 (m, 1H), 7.36-7.40 (m, 1H), 3.17-3.21 (m, 1H), 2.88-3.02 (m, 2H), 2.65-2.68 (m, 1H), 2.16-2.35 (m, 1H), 1.99-2.04 (m, 1H), 1.57-1.60 (m, 1H), 1.38 (s, 3H), 0.66 (d, J=7.05 Hz, 3H); MS (M+1)=233.2.

### Preparation 5

### (+/−)-3-(1-Benzyl-trans-3,4-dimethyl-piperidin-4-yl)-benzoic acid methyl ester

To a solution of (+/−)-trifluoro-methanesulfonic acid 3-(1-benzyl-trans-3,4-dimethyl-piperidin-4-yl)-phenyl ester [EP 1072592] (12.2 g, 28.5 mmol) in a Parr pressure bottle in MeOH (45 mL) were added DMSO (21 mL) and triethylamine (25 mL). To the reaction mixture was added palladium acetate (4.47 g, 19.9 mmol) and 1,3-bis(diphenylphosphino) propane (5.88 g, 14.3 mmol). The mixture was shaken under 40 psi of CO at 70° C. for 16 hours. The reaction mixture was cooled to room temperature and the solvent was removed under reduced pressure. Resulting material was taken up in ethyl acetate and water and filtered through celite. The layers were separated and the aqueous layer was extracted with ethyl acetate. The combined organic layers were washed with brine solution, dried (MgSO₄) and concentrated. The crude residue was purified by flash chromatography with hexanes/EtOAc (1:1) to afford 5.0 g (60% yield) of the desired product. ¹HNMR (400 MHz, CDCl₃) δ 7.94-7.95 (m, 1H), 7.81-7.83 (m, 1H), 7.44-7.47 (m, 1H), 7.19-7.37 (m, 6H), 3.89 (s, 3H), 3.50 (ABq, ΔAB=53.1 Hz, J=13.3 Hz, 2H), 2.82-2.85 (m., 1H), 2.52-2.57 (m, 2H), 2.36-2.39 (m, 2H), 2.00-2.02 (m, 1H), 1.61-1.63 (m, 1H), 1.31 (s, 3H), 0.73 (d, J=7.05 Hz, 3H); MS (M+1) 338.2.

### Preparation 6

### (+/−)-3-(trans-3,4-Dimethyl-piperidin-4-yl)-benzoic acid methyl ester

In a Parr bottle, (+/−)-3-(1-Benzyl-trans-3,4-dimethyl-piperidin-4-yl)-benzoic acid methyl ester (prepared as described above, 3.0 g, 8.9 mmol) was dissolved in 110 mL methanol at room temperature. To this solution was added 700 mg of 10% Pd(C). The mixture was hydrogenated under 50 psi H₂ at 60° C. for 24 hours. The mixture was cooled to room temperature and filtered through a plug of celite and the pad was washed several times with methanol. The resulting solution was concentrated under reduced pressure to yield 2.2 g of the desired product. 400 MHz ¹H NMR (CDCl₃) δ 7.91-7.92 (m, 1H), 7.83-7.86 (m, 1H), 7.42-7.45 (m, 1H), 7.35-7.39 (m, 1H), 3.88 (s, 3H), 3.27-3.31 (m, 1H), 3.17-3.20 (m, 1H), 3.01-3.08 (m, 1H), 2.81-2.85 (m, 1H), 2.23-2.30 (m, 1H), 2.05-2.07 (m, 1H), 1.65-1.68 (m, 1H), 1.38 (s, 3H), 0.73 (d, J=7.05 Hz, 3H); MS (M+1) 248.2.

### Preparation 7

### (+/−)-3-{1-[3-(1-propyl]-trans-3,4-dimethyl-piperidin-4-yl}-benzoic acid methyl ester

To a stirring solution of (+/−)-3-(trans-3,4-Dimethyl-piperidin-4-yl)-benzoic acid methyl ester (900 mg, 3.64 mmol) in 5 mL dichloromethane and 3 mL methanol, was added 3-(1-Hydroxy-cyclohexyl)-propionaldehyde (853 mg, 5.47 mmol) and sodium triacetoxyborohydride (1.16 g, 5.47 mmol). The reaction mixture was stirred at room temperature for 3 hours. The mixture was quenched by the addition of saturated sodium bicarbonate solution and extracted with methylene chloride. The combined organic layers were dried over anhydrous MgSO, and concentrated under reduced pressure. The resulting crude material was purified by flash chromatography with 75% ethyl acetate/hexanes. The product containing fractions were collected and concentrated to yield 923 mg of the desired product. 400 MHz ¹H NMR (CDCl₃) 7.92-7.93 (m, 1H), 7.81-7.83 (m, 1H), 7.42-7.45 (m, 1H), 7.32-7.36 (m, 1H), 3.88 (s, 3H), 2.88-2.90 (m, 1H), 2.66-2.69 (m, 1H), 2.47-2.50 (m, 1H), 2.31-2.39 (m, 4H), 2.04-2.07 (m, 1H), 1.31-1.65 (m, 18H), 0.72 (d, J=7.05 Hz, 3H); MS (M+1) 388.2.

### Example 1

### (+/−)-3-{1-[3-(1-Hydroxy-cyclohexyl)-propyl]-trans-3,4-dimethyl-piperidin-4-yl}-N-(2-methoxy-ethyl)-benzamide

To a solution of 2-Methoxy-ethylamine (76.5 μL, 0.88 mmol) in ClCH₂CH₂Cl (2 mL) at room temperature was added a trimethylaluminum (440 μL, 0.88 mmol, 2M in hexanes) dropwise. The reaction mixture was stirred at room temperature for 1 hour. A solution of of (+/−)-3-{[1 (1-Hydroxy-cyclohexyl)-propyl]-trans-3,4-dimethyl-piperidin-4-yl}-benzoic acid methyl ester (62 mg, 0.16 mmol) in (CH₂)₂Cl₂ (2 mL) was added and the reaction mixture was heated to reflux for 24 hours. The solution was then cooled 0° C. and saturated aqueous NaHCO₃ and saturated sodium potassium tartrate solution was added dropwise. The aqueous was extracted with CH₂Cl₂. The combined organic layers were dried (MgSO₄) and concentrated. The crude residue was purified by flash chromatography with 50% ethyl acetate/hexanes to afford 49 mg (71% yield) of the desired product. ¹HNMR (400 MHz, CDCl₃) δ 7.70 (s, 1H), 7.48-7.50 (m, 1H), 7.23-7.38 (m, 2H), 6.47 (brs, 1H), 3.61-3.63 (m, 2H), 3.53-3.55 (m, 2H), 3.36 (s, 3H), 2.87-2.89 (m, 1H), 2.66-2.69 (m, 1H), 2.46-2.50 (m, 1H), 2.31-2.38 (m, 4H), 2.02-2.06 (m, 1H), 1.31-1.61 (m, 18H), 0.73 (d, J=7.05 Hz, 3H); MS (M+1) 431.3

The following compounds were prepared using the procedure above in example 1.

CP-841724-10: (+/−)-3-{1-[3-(1-Hydroxy-cyclohexyl)-propyl]-trans-3,4-dimethyl-piperidin-4-yl}-N-methyl-benzamide

¹HNMR (400 MHz, CDCl₃) δ 7.66-7.67 (m, 1H), 7.48-7.51 (m, 1H), 7.29-7.38 (m, 2H), 6.23 (brs, 1H), 2.99, 2.98 (two-s, 3H total), 2.81-2.85 (m, 1H), 2.66-2.69 (m, 1H), 2.41-2.49 (m, 1H), 2.30-2.38 (m, 4H), 2.03-2.05 (m, 1H), 1.20-1.64 (m, 18H), 0.71 (d, J=7.05 Hz, 3H); MS (M+1) 387.2.

CP-841725-10: (+/−)-3-{1-[3-(1-Hydroxy-cyclohexyl)-propyl]-trans-3,4-dimethyl-piperidin-4-yl}-N-(tetrahydro-furan-2-ylmethyl)-benzamide

¹HNMR (400 MHz, CDCl₃) δ 7.70 (s, 1H), 7.48-7.50 (m, 1H), 7.23-7.36 (m, 2H), 6.57 (brs, 1H), 4.01-4.07 (m, 1H), 3.83-3.86 (m, 1H), 3.70-3.76 (m, 2H), 3.28-3.32 (m, 1H), 2.80-2.84 (m, 1H), 2.63-2.65 (m, 1H), 2.45-2.48 (m, 1H), 2.32-2.37 (m, 4H), 1.84-2.02 (m, 5H), 1.20-1.61 (m, 18H), 0.70 (d, J=7.05 Hz, 3H); MS (M+1) 457.3.

### Example 2

### General Procedure for the Reductive Alkylation Preparation of Compounds of Formula (IX)

To a stirring solution of 1.0 equiv. of a compound of formula (VII) in methylene chloride (0.2 M) at room temperature was added an aldehyde of formula (VIII) (2.0 equiv.), acetic acid (2.0 equiv.) and sodium triacetoxyborohydride (2.0 equiv.). The reaction mixtures were stirred at room temperature for up to 24 hours. The mixtures were then quenched by the addition of saturated sodium bicarbonate solution and extracted with methylene chloride. The combined organic layers were dried over anhydrous MgSO₄ and concentrated under reduced pressure. The resulting crude material was purified by flash chromatography to yield the desired tertiary amines of formula (IX) in 30-90% yield.

The following compounds were made using the above procedure of Example 2, starting with the appropriate starting amine of formula (VII) and the appropriate aldehyde reagent of formula (VIII).

CP-759039-01: (+/−)-3-(trans-3,4-Dimethyl-1-phenethyl-piperidin-4-yl)-benzamide

¹HNMR (400 MHz, CDCl₃) δ 7.78-7.79 (m, 1H), 7.53-7.55 (m, 1H), 7.44-7.46 (m, 1H), 7.27-7.38 (m, 1H), 7.14-7.27 (m, 5H), 6.10 (brs, 1H), 5.85 (brs, 1H), 2.86-2.88 (m, 1H), 2.74-2.80 (m, 2H), 2.51-2.66 (m, 4H), 2.34-2.42 (m, 2H), 2.02-2.07 (m, 1H), 1.64-1.67 (m, 1H), 1.32 (s, 3H), 0.74 (d, J=7.05 Hz, 3H); MS (M+1) 337.3.

CP-761055: (+/−)-3-{1-[3-(1-Hydroxy-cyclohexyl)-propyl]-trans-3,4-dimethyl-piperidin-4-yl}-benzamide

¹HNMR (400 MHz, CDCl₃) δ 7.74 (s, 1H), 7.54-7.56 (m, 1H), 7.41-7.42 (m, 1H), 7.34-7.40 (m, 1H), 6.25 (brs, 1H), 5.80 (brs, 1H), 2.86-2.89 (m, 1H), 2.66-2.69 (m, 1H), 2.47-2.50 (m, 1H), 2.27-2.42 (m, 4H), 2.05-2.07 (m, 1H), 1.15-1.64 (m, 18H), 0.72 (d, J=7.05 Hz, 3H); MS (M+1) 373.4.

CP-777263: (+)-3-{1-[3-(1-Hydroxy-cyclohexyl)-propyl]-trans-3,4-dimethyl-piperidin-4-yl}-benzamide

MS (M+1) 373.3; [α]_{D}+48.20 (c 0.50, CHCl₃).

CP-777509: (−)-3-{1-[3-(1-Hydroxy-cyclohexyl)-propyl]-trans-3,4-dimethyl-piperidin-4-yl}-benzamide

MS (M+1) 373.3; [α]_{D}−40.9° (c 0.57, CHCl₃).

CP-803241-10: (+/−)-3-{1-[3-(1-Hydroxy-cyclopentyl)-propyl]-trans-3,4-dimethyl-piperidin-4-yl}-benzamide

¹HNMR (400 MHz, CDCl₃) δ 7.74 (s, 1H), 7.54-7.56 (m, 1H), 7.41-7.42 (m, 1H), 7.34-7.40 (m, 1H), 6.25 (brs, 1H), 5.80 (brs, 1H), 2.86-2.89 (m, 1H), 2.66-2.69 (m, 1H), 2.47-2.50 (m, 1H), 2.27-2.42 (m, 4H), 2.05-2.07 (m, 1H), 1.15-1.64 (m, 18H), 0.72 (d, J=7.05 Hz, 3H); MS (M+1) 373.4

CP-803446-10: (+/−)-3-{1-[3-(1-Methoxy-cyclohexyl)-propyl]-trans-3,4-dimethyl-piperidin-4-yl)-benzamide

¹HNMR (400 MHz, CDCl₃) δ 7.76-7.77 (m, 1H), 7.52-7.54 (m, 1H), 7.42-7.44 (m, 1H), 7.32-7.38 (m, 1H), 6.10 (brs, 1H), 5.79 (brs, 1H), 3.10 (s, 3H), 2.80-2.82 (m, 1H), 2.56-2.59 (m, 1H), 2.45-2.49 (m, 1H), 2.32-2.38 (m, 3H), 2.22-2.27 (m, 1H), 2:02-2.04 (m, 1H), 1.61-1.68 (m, 3H), 1.31-1.54 (m, 10H), 1.30 (s, 3H), 1.19-1.23 (m, 2H), 0.72 (d, J=7.05 Hz, 3H); MS (M+1) 387.2.

CP-820213-10: (+/−)-3-{1-[3-(1-Hydroxymethyl-cyclopentyl)-propyl]-trans-3,4-dimethyl-piperidin-4-yl}-benzamide

¹HNMR (400 MHz, CDCl₃) δ 7.75-7.76 (m, 1H), 7.52-7.55 (m, 1H), 7.42-7.44 (m, 1H), 7.32-7.36 (m, 1H), 6.10 (brs, 1H), 5.70 (brs, 1H), 3.34 (ABq, ΔAB=26.8 Hz, J=11.0 Hz, 2H), 2.80-2.82 (m, 1H), 2.56-2.57 (m, 1H), 2.42-2.49 (m, 1H), 2.27-2.41 (m, 4H), 2.02-2.04 (m, 1H), 1.61-1.63 (m, 1H), 1.31-1.57 (m, 15H), 0.73 (d, J=7.05 Hz, 3H); MS (M+1) 373.3.

CP-835922-10: (+)-3-{1-[3-(1-Hydroxymethyl-cyclopentyl)-propyl]-trans-3,4-dimethyl-piperidin-4-yl}-benzamide

MS (M+1) 373.3; [α]D+48.4° (c 0.41, CHCl₃).

CP-835926-10: (−)-3-{1-[3-(1-Hydroxymethyl-cyclopentyl)-propyl]-trans-3,4-dimethyl-piperidin-4-yl}-benzamide

MS (M+1) 373.3; [α]_{D} −46.7° (c 0.46, CHCl₃).

CE-156401-10: (+)-3-{1-[2-(1H-Inden-2-yl)-ethyl]-trans-3,4-dimethyl-piperidin-4-yl}-benzemide

¹HNMR (400 MHz, CDCl₃) δ 7.78 (s, 1H), 7.53-7.55 (m, 1H), 7.42-7.44 (m, 1H), 7.33-7.37 (m, 2H), 7.23-7.25 (m, 1H), 7.16-7.20 (m, 1H), 7.05-7.09 (m, 1H), 6.53 (s, 1H), 6.18 (brs, 1H), 5.93 (brs, 1H), 3.33 (s, 2H), 2.90-2.95 (m, 1H), 2.61-2.69 (m, 5H), 2.33-2.46 (m, 3H), 2.02-2.08 (m, 1H), 1.67-1.70 (m, 1H), 1.32 (s, 3H), 0.74 (d, J=7.05 Hz, 3H); MS (M+1) 375.3.

CE-156402-10: (+)-(3-{1-[2-(2-Hydroxy-indan-2-yl)-ethyl]-trans-3,4-dimethyl-piperidin-4-yl}-benzamide

¹HNMR (400 MHz, CDCl₃) δ 7.73 (s, 1H), 7.52-7.54 (m, 1H), 7.31-7.38 (m, 2H), 7.08-7.17 (m, 4H), 6.27 (brs, 1H), 5.69 (brs, 1H), 3.09-3.13 (m, 1H), 2.93-3.06 (m, 4H), 2.68-2.77 (m, 4H), 2.32-2.42 (m, 2H), 2.03-2.09 (m, 1H), 1.93-1.99 (m, 1H), 1.80-1.85 (m, 1H), 1.67-1.70 (m, 1H), 1.32 (s, 3H), 0.66 (d, J=7.05 Hz, 3H); MS (M+1) 393.2.

CE-157623-10: (+)-3-(1-{2-[3-(1-Hydroxy-cyclohexyl)-phenyl]-ethyl}-trans-3,4-dimethyl-piperidin-4-yl)-benzamide

¹HNMR (400 MHz, CDCl₃) δ 7.77-7.79 (m, 1H), 7.52-7.55 (m, 1H), 7.42-7.44 (m, 1H), 7.28-7.36 (m, 3H), 7.21-7.24 (m, 1H), 7.07-7.09 (m, 1H), 6.20 (brs, 1H), 6.02 (brs, 1H), 2.73-2.89 (m, 3H), 2.51-2.67 (m, 4H), 2.31-2.46 (m, 2H), 2.06-2.08 (m, 1H), 1.60-1.84 (m, 10H), 1.31 (s, 3H), 1.21-1.28 (m, 1H), 0.73 (d, J=7.05 Hz, 3H); MS (M+1) 435.3

CE-157632-10: (+)-3-[1-(cis-1-Hydroxy-3-phenyl-cyclobutylmethyl)-trans-3,4-dimethyl-piperidin-4-yl]-benzamide

¹HNMR (400 MHz, CDCl₃) δ 7.77 (s, 1H), 7.54-7.56 (m, 1H), 7.35-7.43 (m, 2H), 7.23-7.29 (m, 4H), 7.14-7.18 (m, 1H), 6.16 (brs, 1H), 5.83 (brs, 1H), 2.87-3.05 (m, 3H), 2.65-2.72 (m, 4H), 2.47-2.55 (m, 2H), 2.33-2.40 (m, 3H), 2.01-2.10 (m, 1H), 1.67-1.70 (m, 1H), 1.35 (s, 3H), 0.76 (d, J=7.05 Hz, 3H); MS (M+1) 393.3.

CE-187319-10: (+)-2-{2-[4-(3-Carbamoyl-phenyl)-trans-3,4-dimethyl-piperidin-1-yl]-ethyl}-indan-2-carboxylic acid tert-butyl ester

¹HNMR (400 MHz, CDCl₃) δ 7.78 (s, 1H), 7.55-7.57 (m, 1H), 7.43-7.45 (m, 1H), 7.35-7.39 (m, 1H), 7.11-7.17 (m, 4H), 6.17 (brs, 1H), 5.91 (brs, 1H), 3.41 (dd, J=16.1, 5.4 Hz, 2H), 2.92 (d, J=16.1 Hz, 2H), 2.81 (brs, 1H), 2.39-2.55 (m, 2H), 2.33-2.35 (m, 4H), 1.99-2.00 (m, 1H), 1.88-1.97 (m, 2H), 1.59-1.66 (m, 1H), 1.44 (s, 9H), 1.31 (s, 3H), 0.73 (d, J=6.60 Hz, 3H); MS (M+1) 477.4.

CE-190738-51: (+)-2-{2-[4-(3-Carbamoyl-phenyl)-trans-3,4-dimethyl-piperidin-1-yl]-ethyl}-indan-2-carboxylic acid

¹HNMR (400 MHz, CD₃OD) δ 7.88 (s, 1H), 7.70-7.72 (m, 1H), 7.41-7.48 (m, 2H), 7.09-7.19 (m, 4H), 3.43-3.59 (m, 4H), 3.26-3.40 (m, 4H), 3.00 (dd, J=16.2, 3.32 Hz, 2H), 2.43-2.49 (m, 2H), 2.22-2.30 (m, 1H), 2.10-2.19 (m, 1H), 1.98-2.01 (m, 1H), 1.45 (s, 3H), 0.73 (d, J=6.90 Hz, 3H); MS (M+1) 421.3.

CE-191385-01: (+)-2-{2-[4-(3-Carbamoyl-phenyl)-trans-3,4-dimethyl-piperidin-1-yl]-ethyl}-indan-2-carboxylic acid amide

¹HNMR (400 MHz, CD₃OD) δ 7.83 (s, 1H), 7.67-7.69 (m, 1H), 7.47-7.49 (m, 1H), 7.36-7.40 (m, 1H), 7.09-7.18 (m, 4H), 3.34-3.39 (m, 2H), 2.95 (d, J=15.9 Hz, 2H), 2.81-2.84 (m, 1H), 2.61-2.62 (m, 2H), 2.29-2.47 (m, 4H), 2.01-2.13 (m, 1H), 1.83-2.01 (m, 2H), 1.61-1.69 (m, 1H), 1.32 (s, 3H), 0.71 (d, J=7.05 Hz, 3H); MS (M+1) 420.4

CE-215811-01: (+)-3-{trans-3,4-Dimethyl-1-[3-(2-nitro-indan-2-yl)-propyl]-piperidin-4-yl}-benzamide

¹HNMR (400 MHz, CDCl₃) δ 7.75 (s, 1H), 7.52-7.54 (m, 1H), 7.41-7.43 (m, 1H), 7.33-7.37 (m, 1H), 7.13-7.18 (m, 4H), 6.09 (brs, 1H), 5.71 (brs, 1H), 3.85 (d, J=17.1 Hz, 2H), 3.20 (dd, J=17.1, 4-0.56 Hz, 2H), 2.77 (brs, 1H), 2.50 (s, 2H), 2.32-2.36. (m, 4H), 2.12-2.18 (m, 2H), 2.04-2.11 (m, 1H), 1.64 (brs, 1H), 1.45-1.49 (m, 2H), 1.29 (s, 3H), 0.70 (d, J=6.60 Hz, 3H); MS (M+1) 436.4.

CE-223922-01: (+)-3-{1-[3-(2-Amino-indan-2-yl)-propyl]-trans-3,4-dimethyl-piperidin-4-yl}-benzamide

¹HNMR (400 MHz, CDCl₃) δ 7.74 (s, 1H), 7.51-7.55 (m, 1H), 7.41-7.43 (m, 1H), 7.32-7.36 (m, 1H), 7.09-7.15 (m, 4H), 6.37 (brs, 1H), 5.85 (brs, 1H), 2.97 (d, J=15.8 Hz, 2H), 2.77-2.87 (m, 3H), 2.32-2.62 (m, 9H), 2.02-2.04 (m, 1H), 1.60-1.69 (m, 4H), 1.30 (s, 3H), 0.70 (d, J=7.05 Hz, 3H); MS (M+1) 406.4.

CE-255265-10: (+)-3-{1-[cis-3-(4-Bromo-phenyl)-1-hydroxy-cyclobutylmethyl]-trans-3,4-dimethyl-piperidin-4-yl}-benzamide.

¹HNMR (400 MHz, CDCl₃) δ 7.77 (s, 1H), 7.54-7.55 (m, 1H), 7.35-7.43 (m, 4H), 7.09 (d, J=8.3 Hz, 2H), 6.10 (brs, 1H), 5.66 (brs, 1H), 2.71-3.00 (m, 6H), 2.29-2.54 (m, 5H), 2.05-2.12 (m, 1H), 1.60-1.69 (m, 2H), 1.35 (s, 3H), 0.76-0.78 (m, 3H); MS (M+1) 473.3.

CE-255272-10: (+)-3-{1-[cis-1-Hydroxy-3-(4-methoxy-phenyl)-cyclobutylmethyl]-trans-3,4-dimethyl-piperidin-4-yl}-benzamide

¹HNMR (400 MHz, CDCl₃) δ 7.78 (s, 1H), 7.54-7.56 (m, 1H), 7.35-7.43 (m, 2H), 7.14-7.16 (m, 2H), 6.81-6.84 (m, 2H), 6.15 (brs, 1H), 5.65 (brs, 1H), 3.76 (s, 3H), 2.81-2.98 (m, 6H), 2.48-2.53 (m, 3H, 2.31-2.36 (m, 2H), 2.10-2.19 (m, 1H), 1.83-1.91 (m, 2H), 1.36 (s, 3H), 0.81 (brs, 3H); MS (M+1) 423.4.

CE-263237-01: (+)-3-{1-[2-(2-Amino-indan-2-yl)-ethyl]-trans-3,4-dimethyl-piperidin-4-yl}-benzamide

¹HNMR (400 MHz, CDCl₃) δ 7.51 (brs, 1H), 7.38-7.46 (m, 2H), 7.22 (s, 1H), 7.04-7.20 (m, 5H), 6.76 (brs, 1H), 3.02-3.35 (m, 6H), 2.64-2.75 (m, 4H), 2.15-2.34 (m, 4H), 1.88-1.92 (m, 2H), 1.48-1.51 (m, 1H), 1.24 (s, 3H), 0.42 (d, J=7.05 Hz, 3H); MS (M+1) 392.4.

CE-263490-01: (+)-3-{1-[2-(2-Acetylamino-indan-2-yl)-ethyl]-trans-3,4-dimethyl-piperidin-4-yl}-benzamide

¹HNMR (400 MHz, CDCl₃) δ 7.81 (s, 1H), 7.78 (brs, 1H); 7.54-7.55 (m, 1H), 7.41-7.43 (m, 1H), 7.34-7.38 (m, 1H), 7.07-7.12 (m, 4H), 6.27 (brs, 1H), 5.81 (brs, 1H), 3.66-3.72 (m, 2H), 2.97-3.00 (m, 1H), 2.71-2.80 (m, 3H), 2.34-2.59 (m, 5H), 2.13-2.14 (m, 1H), 1.87 (s, 5H), 1.73-1.76 (m, 1H), 1.32 (s, 3H), 0.78 (d, J=7.05 Hz, 3H); MS (M+1) 434.4.

CE-190738: (+)-2-12-[4-(3-Carbamoyl-phenyl)-trans-3,4-dimethyl-piperidin-1-yl]-ethyl}-indan-2-carboxylic acid-TFA salt

¹HNMR (400 MHz, CD₃OD) δ 7.79 (s, 1H), 7.70-7.72 (m, 1H), 7.41-7.50 (m, 2H), 7.09-7.19 (m, 4H), 3.24-3.59 (m, 8H), 3.00 (d, J=16.2 Hz, 2H), 2.43-2.51 (m, 2H), 2.2072.28 (m, 1H), 2.09-2.17 (m, 1H), 1.95-2.02 (m, 1H), 1.45 (s, 3H), 0.71 (d, J=7.05 Hz, 3H); MS (M+1) 421.2.

### Example 3

### General Procedure for the Preparation of Compounds of Formula (IX)

To a stirring solution of a compound of formula (VII) in dimethylformamide (0.1 M) at room temperature was added sodium bicarbonate (4 equiv.) and the appropriate reagent of formula (X) (1.1 equiv.). The resulting mixture is heated to 80° C. for 1-8 hours and then cooled to room temperature. The mixture was partitioned between ethyl acetate and 1N LiCl solution, the layers were separated, and the organic layer was washed several times with water and brine solution. The organic layer was dried over anhydrous MgSO₄ and concentrated under reduced pressure. The crude material was purified by flash chromatography to yield the desired product of formula (IX) in 35-75% yield.

The following compounds were made using the above procedure of Example 3, starting with the appropriate starting amine of formula (VII) and the appropriate reagent of formula (X).

CP-759901-01: (+/−)-3-(1-Indan-2-ylmethyl-trans-3,4-dimethyl-piperidin-4-yl)-benzamide

¹HNMR (400 MHz, CDCl₃) δ 7.77 (s, 1H), 7.53-7.55 (m, 1H), 7.44-7.46 (m, 1H), 7.35-7.38 (m, 1H), 7.16-7.18 (m, 2H), 7.08-7.11 (m, 2H), 6.06 (brs, 1H), 5.69 (brs, 1H), 2.95-3.04 (m, 2H), 2.82-2.83 (m, 1H), 2.63-2.74 (m, 2H), 2.51-2.59 (m, 2H), 2.28-2.39 (m, 4H), 2.02-2.04 (m, 1H), 1.61-1.63 (m, 2H), 1.31 (s, 3H), 0.75 (d, J=7.05 Hz, 3H); MS (M+1) 363.3.

CP-767878-01: (+/−)-3-(1-Imidazo[1,2-a]pyridin-2-ylmethyl-trans-3,4-dimethyl-piperidin-4-yl)-benzamide

¹HNMR (400 MHz, CDCl₃) δ 8.03-8.05 (m, 1H), 7.76-7.77 (m, 1H), 7.48-7.55 (m, 3H), 7.42-7.44 (m, 1H), 7.32-7.36 (m, 1H), 7.07-7.11 (m, 1H), 6.69-6.73 (m, 1H), 6.20 (brs, 1H), 5.78 (brs, 1H), 3.72 (ABq, ΔAB=38.2 Hz, J=14.1 Hz, 2H), 2.93-2.95 (m, 1H), 2.65-2.67 (m, 2H), 2.48-2.52 (m, 1H), 2.37-2.39 (m, 1H), 2.02-2.04 (m, 1H), 1.61-1.63 (m, 1H), 1.30 (s, 3H), 0.75 (d, J=7.05 Hz, 3H); MS (M+1) 363.3.

CP-774879-01: (+/−)-3-{1-[2-(4-Methoxy-phenyl)-ethyl]-trans-3,4-dimethyl-piperidin-4-yl}-benzamide

¹HNMR (400 MHz, CDCl₃) δ7.77-7.79 (m, 1H), 7.52-7.54 (m, 1H), 7.43-7.46 (m, 1H), 7.34-7.38 (m, 1H—), 7.10-7.13 (m, 2H), 6.78-6.82 (m, 2H), 6.05 (brs, 1H), 5.62 (brs, 1H), 3.76 (s, 3H), 2.85-2.88 (m, 1H), 2.34-2.74 (m, 8H), 2.05-2.07 (m, 1H), 1.63-1.64 (m, 1H), 1.31 (s, 3H), 0.74 (d, J=7.05 Hz, 3H); MS (M+1) 367.3.

CP-775356-01: (+/−)-3-{1-[2-(2-Methoxy-phenyl)-ethyl]-trans-3,4-dimethyl-piperidin-4-yl}-Benzamide

¹HNMR (400 MHz, CDCl₃) δ 7.78-7.79 (m, 1H), 7.53-7.55 (m, 1H), 7.45-7.47 (m, 1H), 7.35-7.39 (m, 1H), 7.14-7.18 (m, 2H), 6.82-6.88 (m, 2H), 6.09 (brs, 1H), 5.69 (brs, 1H), 3.81 (s, 3H), 2.32-2.89 (m, 9H), 2.02-2.07 (m, 1H), 1.65-1.68 (m, 1H), 1.33 (s, 3H), 0.75 (d, J=7.05 Hz, 3H); MS (M+1) 367.3.

CP-775358-01: (+/−)-3-{1-[2-(3-Methoxy-phenyl)-ethyl]-trans-3,4-dimethyl-piperidin-4-yl}-benzamide

¹HNMR (400 MHz, CDCl₃) δ 7.78-7.79 (m, 1H), 7.54-7.56 (m, 1H), 7.44-7.47 (m, 1H), 7.35-7.39 (m, 1H), 7.16-7.20 (m, 1H), 6.77-6.81 (m, 2H), 6.71-6.74 (m, 1H), 6.10 (brs, 1H), 5.70 (brs, 1H), 3.78 (s, 3H), 2.87-2.89 (m, 1H), 2.35-2.79 (m, 8H), 2.06-2.08 (m, 1H), 1.65-1.68 (m; 1H), 1.33 (s, 3H), 0.75 (d, J=7.05 Hz, 3H); MS (M+1) 367.3.

CP-777250-01: (+/−)-3,4-{trans-3,4-Dimethyl-1-[2-(3-trifluoromethyl-phenyl)-ethyl]-piperidin-4-yl}-benzamide

¹HNMR (400 MHz, CDCl₃) δ 7.76-7.78 (m, 1H), 7.52-7.54 (m, 1H), 7.33-7.50 (m, 6H), 6.05 (brs, 1H), 5.67 (brs, 1H), 2.76-2.89 (m, 3H), 2.51-2.66 (m, 4H), 2.29-2.44 (m, 2H), 2.02-2.07 (m, 1H), 1.63-1.67 (m, 1H), 1.32 (s, 3H), 0.71 (d, J=7.05 Hz, 3H); MS (M+1) 405.2.

CP-777252-01: (+/−)-3-{1-[2-(4-Cyano-phenyl)-ethyl]-trans-3,4-dimethyl-piperidin-4-yl}-benzamide

¹HNMR (400 MHz, CDCl₃) δ 7.77-7.78 (m, 1H), 7.51-7.54 (m, 3H), 7.42-7.44 (m, 1H), 7.29-7.38 (m, 3H), 6.07 (brs, 1H), 5.61 (brs, 1H), 2.79-2.86 (m, 3H), 2.50-2.65 (m, 4H), 2.28-2.44 (m, 2H), 2.02-2.06 (m, 1H), 1.63-1.71 (m, 1H), 1.31 (s, 3H), 0.68 (d, J=7.05 Hz, 3H); MS (M+1) 362.2.

CP-781909-01: (+/−)-3-{1-[2-(3-Bromo-phenyl)-ethyl]-trans-3,4-dimethyl-piperidin-4-yl}-benzamide

¹HNMR (400 MHz, CDCl₃) δ 7.77-7.78 (m, 1H), 7.52-7.54 (m, 1H), 7.43-7.45 (m, 1H), 7.34-7.38 (m, 2H), 7.27-7.30 (m, 1H), 7.09-7.13 (m, 2H), 6.10 (brs, 1H), 5.71 (brs, 1H), 2.84-2.88 (m, 1H), 2.67-2.78 (m, 2H), 2.43-2.62 (m, 4H), 2.30-2.40 (m, 2H), 2.05-2.06 (m, 1H), 1.64-1.66 (m, 1H), 1.31 (s, 3H), 0.72 (d, J=7.05 Hz, 3H); MS (M+1) 415.1, 417.1.

CP-781910-01: (+/−)-3-{1-[2-(4-Chloro-phenyl)-ethyl]-trans-3,4-dimethyl-piperidin-4-yl}-benzamide

¹HNMR (400 MHz, CDCl₃) δ 7.77-7.78 (m, 1H), 7.52-7.54 (m, 1H), 7.43-7.45 (m, 1H), 7.34-7.38 (m, 1H), 7.19-7.23 (m, 2H), 7.11-7.14 (m, 2H), 6.08 (brs, 1H), 5.73 (brs, 1H), 2.84-2.88 (m, 1H), 2.67-2.78 (m, 2H), 2.46-2.62 (m, 4H), 2.29-2.43 (m, 2H), 2.05-2.06 (m, 1H), 1.63-1.66 (m, 1H), 1.31 (s, 3H), 0.71 (d, J=7.05 Hz, 3H); MS (M+1) 371.2.

CP-781911-01: (+/−)-3-{1-[2-(3-Chloro-phenyl)-ethyl]-trans-3,4-dimethyl-piperidin-4-yl}-benzamide

¹HNMR (400 MHz, CDCl₃) δ 7.77-7.78 (m, 1H), 7.52-7.55 (m, 1H), 7.42-7.45 (m, 1H), 7.33-7.37 (m, 1H), 7.06-7.20 (m, 4H), 6.10 (brs, 1H), 5.75 (brs, 1H), 2.85-2.87 (m, 1H), 2.71-2.77 (m, 2H), 2.51-2.60 (m, 4H), 2.32-2.40 (m, 2H), 2.04-2.06 (m, 1H), 1.63-1.66 (m, 1H), 1.31 (s, 3H), 0.71 (d, J=7.05 Hz, 3H); MS (M+1) 371.2.

CP-789545-01: (+/−)-3-{1-[2-(3-Cyano-phenyl)-ethyl]-trans-3,4-dimethyl-piperidin-4-yl}-benzamide

¹HNMR (400 MHz, CDCl₃) δ 7.76-7.78 (m, 1H), 7.50-7.54 (m, 2H), 7.42-7.46 (m, 3H), 7.32-7.37 (m, 2H), 6.10 (brs, 1H), 5.71 (brs, 1H), 2.74-2.87 (m, 3H), 2.49-2.64 (m, 4H), 2.28-2.43 (m, 2H), 2.02-2.06 (m, 1H), 1.63-1.66 (m, 1H), 1.31 (s, 3H), 0.68 (d, J=7.05 Hz, 3H); MS (M+1) 362.2.

CP-789546-01: (+/−)-3-{1-[2-(2,6-Dichloro-phenyl)-ethyl]-trans-3,4-dimethyl-piperidin-4-yl}-benzamide

¹HNMR (400 MHz, CDCl₃) δ 7.77-7.78 (m, 1H), 7.53-7.55 (m, 1H), 7.44-7.46 (m, 1H), 7.34-7.38 (m, 1H), 7.22-7.25 (m, 2H), 7.01-7.05 (m, 1H), 6.10 (brs, 1H), 5.65 (brs, 1H), 3.08-3.14 (m, 2H), 2.93-2.95 (m, 1H), 2.70-2.73 (m, 1H), 2.47-2.64 (m, 4H), 2.35-2.39 (m, 1H), 2.01-2.07 (m, 1H), 1.65-1.68 (m, 1H), 1.32 (s, 3H), 0.73 (d, J=7.05 Hz, 3H); MS (M+1) 405.1, 407.1.

CP-789547-01: (+/−)-3-[trans-3,4-Dimethyl-1-(2-pyridin-2-yl-ethyl)-piperidin-4-yl]-benzamide

¹HNMR (400 MHz, CDCl₃) δ 8.47-8.49 (m, 1H), 7.75-7.76 (m, 1H), 7.52-7.56 (m, 2H), 7.41-7.44 (m, 1H), 7.32-7.36 (m, 1H), 7.16-7.18 (m, 1H), 7.04-7.08 (m, 1H), 6.15 (brs, 1H), 5.81 (brs, 1H), 2.85-2.97 (m, 3H), 2.68-2.76 (m, 2H), 2.60-2.61 (m, 2H), 2.39-2.42 (m, 1H), 2.29-2.31 (m, 1H), 2.02-2.04 (m, 1H), 1.61-1.64 (m, 1H), 1.30 (s, 3H), 0.67 (d, J=6.61 Hz, 3H); MS (M+1) 338.3.

CP-800324: (+/−)-3-[1-(2-Hydroxy-2-phenyl-ethyl)-trans-3,4-dimethyl-piperidin-4-yl]-benzamide

¹HNMR (400 MHz, CDCl₃) δ 7.76-7.79 (m, 1H), 7.52-7.56 (m, 1H), 7.42-7.45 (m, 1H), 7.24-7.39 (m, 6H), 6.10 (brs, 1H), 5.73 (brs, 1H), 4.68-4.75 (m, 1H), 2.83-2.92 (m, 2H), 2.66-2.68 (m, 1H), 2.24-2.56 (m, 5H), 2.02-2.06 (m, 1H), 1.64-1.69 (m, 1H), 1.34 (s, 3H), 0.77-0.80 (m, 3H); MS (M+1) 353.3.

CP-800326-01: (+/−)-3-{1-[3-(1-Cyano-cyclohexyl)-propyl]-trans-3,4-dimethyl-piperidin-4-yl}-benzamide

¹HNMR (400 MHz, CDCl₃) δ 7.75-7.76 (m, 1H), 7.52-7.54 (m, 1H), 7.42-7.45 (m, 1H), 7.33-7.37 (m, 1H), 6.08 (brs, 1H), 5.68 (brs, 1H), 2.78-2.80 (m, 1H), 2.46-2.56 (m, 2H), 2.25-2.40 (m, 4H), 2.02-2.04 (m, 1H), 1.93-1.96 (m, 1H), 1.45-1.72 (m, 11H), 1.30 (s, 3H), 1.12-1.21 (m, 3H), 0.71 (d, J=7.05 Hz, 3H); MS (M+1) 382.3.

### EXAMPLE 4

### General Procedure for the Preparation of Compounds of Formula (IX)

To a stirring solution of a compound of formula. (VII) in ethanol (0.1 M) at room temperature was added triethyl amine (3 equiv.) and the appropriate reagent of formula (XI) (1.2 equiv.). The resulting mixture is heated to 80° C. for 1-5 hours and then cooled to room temperature. The mixture is concentrated under reduced pressure and the resulting crude material was purified by flash chromatography to yield the desired tertiary amines in 40-88% yield.

The following compounds were made using the above procedure of Example 4, starting with the appropriate starting amine of formula (VII) and the appropriate reagent of formula (X).

CP-853909-01: (+/−)-3-[1-(2-Hydroxy-3-phenyl-propyl)-trans-3,4-dimethyl-piperidin-4-yl]-benzamide

MS (M+1) 367.4

CP-867708-10: (+)-3-[1-(2-Hydroxy-indan-2-ylmethyl)-trans-3,4-dimethyl-piperidin-4-yl]-benzamide;

¹HNMR (400 MHz, CDCl₃) δ 7.77-7.78 (m, 1H), 7.52-7.55 (m, 1H), 7.42-7.44 (m, 1H), 7.34-7.38 (m, 1H), 7.11-7.20 (m, 4H), 6.19 (brs, 1H), 5.97 (brs, 1H), 2.90-2.97 (m, 6H), 2.60-2.72 (m, 4H), 2.37-2.44 (m, 1H), 2.03-2.08 (m, 1H), 1.64-1.68 (m, 1H), 1.34 (s, 3H), 0.77 (d, J=7.05 Hz, 3H); MS (M+1) 379.2.

CP-867708-10: (+)-3-[1-(2-Hydroxy-indan-2-ylmethyl)-trans-3,4-dimethyl-piperidin-4-yl]-benzamide mesylate

m.p. 137-139° C.

1. A compound according to formula I: 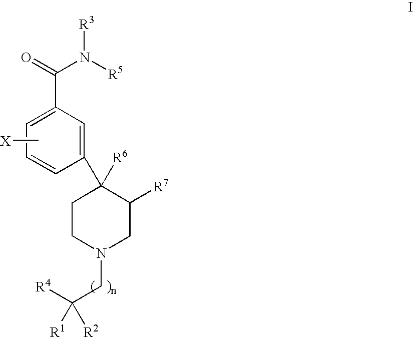
wherein X is H, halogen, or CN;
R¹ and R² are independently H, C₁-C₆ alkyl, —(CH₂)ₖ-aryl, (CH₂)ₖ-heteroaryl, wherein said alkyl, —(CH₂)ₖ-aryl or —(CH₂)ₖ-heteroaryl group is optionally substituted anywhere on said group with one or more R¹² groups, or, with the carbon to which R¹ and R² are attached, are connected to form a C₃-C₇ cycloalkyl or a 4-7 membered carbocyclic or heterocycloalkyl comprising from one to three hetero moities selected from O, S, —C(═O), and N; and wherein said cycloalkyl or heterocycloalkyl optionally contains one or more double bonds; and wherein said cycloalkyl or heterocycloalkyl is optionally fused to or substituted with a C₆-C₁₄ aryl or 5-14 membered heteroaryl group; wherein said C₃-C₇ cycloalkyl or 4-7 membered carbocyclic or heterocycloalkyl formed by R¹ and R² can each optionally be substituted by from one to three R¹² groups, and said optionally fused or substituted aryl or heteroaryl, substituted alkyl, substituted aryl optionally fused aryl or heteroaryl may each optionally independently be substituted with from one to six R¹² groups in any stereochemical relationship;
wherein the R¹² groups are independently selected from H, R¹³, R⁶, —C₁-C₄ alkyl optionally containing one or two unsaturated bonds, halogen, —OR¹³, —NO₂, —CN, —C₃-C₆ cycloalkyl, aryl, substituted aryl, wherein said aryl or substituted aryl is independently optionally substituted with 1-3 R¹⁸ groups, —C(R⁴)(C₁-C₄ alkyl)(C₁-C₄ alkyl) wherein said alkyl groups may form a C₃-C₇ carbocyclic ring, —(CH₂), —NR³R¹⁴, —NR¹³C(═O)R¹⁴, —C(═O)NR¹³R¹⁴, —OC(═O)R¹³, —C(═O)OR¹³, —C(═O)R¹³, —NR¹³C(═O)OR¹⁴, —NR¹³C(═O)NR¹⁴R¹⁵, —NR¹³S(═O)₂R¹⁴, —NR⁷S(═O)₂NR³R¹⁴ and —S(═O)₂R¹³;
R¹⁸ is H, F, Cl, —OH, —C₁-C₄ alkyl, —C═N, —NR¹³C(═O)R¹⁴, C(═O)NR¹³R¹⁴, (C₁-C₄)alkyl, —NH(C₁-C₄ alkyl), —N(C₁-C₄ alkyl C₁-C₄ alkyl), —(CH₂)ₙOH, —(CH₂)—C≡N, —(CH₂)ₙ—NR¹C(═O)R¹⁴, —(CH₂)ₙ—C(═O)NR¹³R¹⁴, (CH₂)ₙ—O(C₁-C₄)alkyl, —(CH₂)ₙ—NH₂—(CH₂)ₙ—NH(C₁-C₄ alkyl) or —(CH₂), —N(C₁-C₄ alkyl)(C₁-C₄ alkyl);
R⁴ is absent or is H, —C₁-C₄ alkyl which may optionally contain one or two unsaturated bonds, —OH, O—(C₁-C₄)alkyl, (C₁-C₄)-alkyl-OH, (CH₂)ₙ—NH₂, —(CH₂)ₙ—NH(C₁-C₄ alkyl), (CH₂)ₙ—N(C₁-C₄)alkyl(C₁-C₄)alkyl, —(CH₂), —NHC(═O)(C₁-C₄ alkyl), —(CH₂)ₙ—NO₂, —(CH₂)ₙ—C═N, —(CH₂), —C(═O)NH₂, —(CH₂), —C(═O)NH(C₁-C₄ alkyl) or —(CH₂)ₙ—C(═O)N(C₁-C₄ alkyl) (C₁-C₄ alkyl), CN, NO₂, —OR¹⁶;
R³ and R⁵ are independently H, alkyl C₁-C₆, substituted alkyl C₁-C₆, cycloalkyl C₁-C₆ and substituted cycloalkyl C₁-C₆, (C₂-C₄)alkyl-O—(C₁-C₄)alkyl, (C₂-C₄)alkyl-NH(C₁-C₄ alkyl), (C₂-C₄)alkyl-N(C₁-C₄ alkyl)(C₁-C₄ alkyl), (C₁-C₄)alkyl-heterocyclic;
R⁶ and R⁷ are independently C₁-C₄ alkyl;
each R¹³, R¹⁴, and R¹⁵ are independently selected from H, —C₁-C₄ alkyl, —(C₂-C₄ alkyl)-O—(C₁-C₄-alkyl), —(CH₂)ᵥ—NR¹⁶R¹⁷, or a 4- to 7-membered heterocyclic group; or R¹³ and R¹⁴ when in —NR¹³R¹⁴, may optionally be connected to form a 4 to 6 membered heterocyclic group, which heterocyclic group optionally comprises from 1 to 3 further hetero moieties selected from N, S, O and —C(═O);
R¹⁶ and R₁₇ are independently H, C₁-C₆ alkyl or together may form a 4- to 7-membered heterocyclic group;
k is an integer selected from zero, 1, 2, 3, 4, and 5; and
v is an integer selected from 2, 3, 4, and 5; and
n is an integer selected from zero, 1, 2, 3, 4, and 5;
and pharmaceutically acceptable salts thereof;
with the proviso that;
a) in said 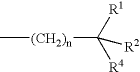
group, when n is 0, R¹, R² or R⁴ cannot be a heteroatom or contain a heteroatom which is directly linked to the carbon of said 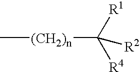
R group when said carbon is sp³ hybridized; and
b) R¹³ and R¹⁴ cannot be H in a —NHS(═O)₂R¹⁴ or a —SO₂R¹³ group.

2. The compound according to claim 1 represented by the chemical structure II: 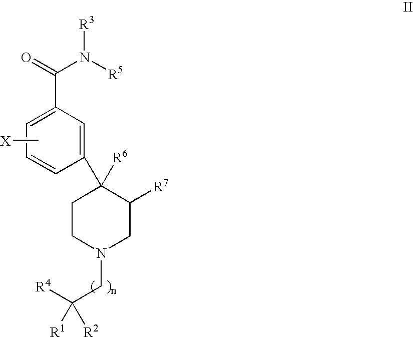
Wherein each of X, R¹, R², R³, R⁴, R⁵, R⁶, R⁷ and n is represented as described in claim 1 and the preferred relative stereochemistry between R⁶ and R⁷ is trans.

3. The compound according to claim 2 wherein R³ and R⁵ are H.

4. The compound according to claim 2 wherein X is H.

5. The compound according to claim 2 wherein R⁶ and R⁷ are each CH₃.

6. The compound according to claim 2 wherein n is 1, 2 or 3.

7. The compound according to claim 2 wherein R is OH, CH₂OH, NH₂, NHCOCH₃ or CN.

8. The compound according to claim 2 wherein R¹ and R² together with the carbon to which they are attached form a carbocyclic group fused to a phenyl group, an unsubstituted or substituted carbocyclic group.

9. The compound according to claim 6 wherein n is 1.

10. The compound according to claim 7 wherein R⁴ is OH.

11. The compound according to claim 2 wherein R¹ and R² together with the carbon to which they are attached form an indane ring system, a cyclobutane, cyclopentane or cyclohexane group.

12. The compound according to claim 2 wherein R¹ and R² together with the carbon to which they are attached form an indane ring system or a cyclobutane group which is substituted with a phenyl group which is unsubstituted or substituted with one or more R¹² groups.

13. The compound according to claim 1 wherein R³ and R⁵ are H, X is hydrogen, R⁶ and R⁷ are CH₃, n is 1, R⁴ is OH, CH₂OH, NH₂, NHCOCH₃ or CN and R¹ and R² together with the carbon to which they are attached, form a carbocyclic group fused to a phenyl ring or an unsubstituted or substituted carbocyclic group.

14. The compound according to claim 2 wherein R³ and R⁵ are H, X is hydrogen, R⁶ and R⁷ are CH₃, n is 1, R⁴ is OH, CH₂OH, NH₂, NHCOCH₃ or CN and R¹ and R² together with the carbon to which they are attached, form a carbocyclic group fused to a phenyl ring or an unsubstituted or substituted carbocyclic group.

15. The compound according to claim 14 wherein R⁴ is OH and R¹ and R² together with the carbon to which they are attached form an indane ring system or a cyclobutane group which is substituted with a phenyl group which is unsubstituted or substituted with one or more R¹² groups.

16. The compound according to claim 14 wherein R⁴ is OH and R¹ and R² together with the carbon to which they are attached form an indane ring system.

17. The compound according to claim 14 wherein R⁴ is OH and R¹ and R² together with the carbon to which they are attached form a cyclobutane group which is substituted with a phenyl group which is unsubstituted or substituted with one or more R¹² groups.

18. The compound:
(+/−)-3-(trans-3,4-Dimethyl-1-phenethyl-piperidin-4-yl)-benzamide;
(+/−)-3-(1-Indan-2-ylmethyl-trans-3,4-dimethyl-piperidin-1-yl)-benzamide;
(+/−)-3-{1-[3-(1-Hydroxy-cyclohexyl)-propyl]-trans-3,4-dimethyl-piperidin-4-yl}-benzamide;
(+/−)-3-{1-[2-(4-Methoxy-phenyl)-ethyl]-trans-3,4-dimethyl-piperidin-4-yl}-benzamide;
(+/−)-3-{1-[2-(2-Methoxy-phenyl)-ethyl]-trans-3,4-dimethyl-piperidin-4-yl}-benzmide;
(+/−)-3-{1-[2-(3-Methoxy-phenyl)-ethyl]-trans-3,4-dimethyl-piperidin-4-yl}-benzamide;
(+/−)-3-{trans-3,4-Dimethyl-1-[2-(3-trifluoromethyl-phenyl)-ethyl]-piperidin-4-yl}-benzamide;
(+/−)-3-{1-[2-(4-Cyano-phenyl)-ethyl]-trans-3,4-dimethyl-piperidin-4-yl}-benzamide;
(+)-3-{1-[3-(1-Hydroxy-cyclohexyl)-propyl]-trans-3,4-dimethyl-piperidin-4-yl}-benzamide;
(−)-3-{1-[3-(1-Hydroxy-cyclohexyl)-propyl]-trans-3,4-dimethyl-piperidin-4-yl}-benzamide;
(+/−)-3-{1-[2-(3-Bromo-phenyl)-ethyl]-trans-3,4-dimethyl-piperidin-4-yl}-benzamide;
(+/−)-3-{1-[2-(4-Chloro-phenyl)-ethyl]-trans-3,4-dimethyl-piperidin-4-yl}-benzamide;
(+/−)-3-{1-[2-(3-Chloro-phenyl)-ethyl]-trans-3,4-dimethyl-piperidin-4-yl}-benzamide;
(+/−)-3-{1-[2-(3-Cyano-phenyl)-ethyl]-trans-3,4-dimethyl-piperidin-4-yl}-benzamide;
(+/−)-3-{1-[2-(2,6-Dichloro-phenyl)ethyl]-trans-3,4-dimethyl-piperidin-4-yl}-benzamide;
(+/−)-3-[trans-3,4-Dimethyl-1-(2-pyridin-2-yl-ethyl)-piperidin-4-yl]-benzamide;
(+/−)-3-[1-(2-Hydroxy-2-phenyl-ethyl)-trans-3,4-dimethyl-piperidin-4-yl]-benzamide;
(+/−)-3-{1-[3-(1-Cyano-cyclohexyl)-propyl]-trans-3,4-dimethyl-piperidin-4-yl}-benzamide;
(+/−)-3-{1-[3-(1-Hydroxy-cyclopentyl)-propyl]-trans-3,4-dimethyl-piperidin-4-yl}-benzamide;
(+/−)-3-{1-[3-(1-Methoxy-cyclohexyl)propyl]-trans-3,4-dimethyl-piperidin-4-yl}-benzamide;
(+/−)-3-{1-[3-(1-Hydroxymethyl-cyclopentyl)propyl]-trans-3,4-dimethyl-piperidin-4-yl}-benzamide;
(+)-3-{1-[3-(1-Hydroxymethyl-cyclopentyl)-propyl]-trans-3,4-dimethyl-piperidin-4-yl}-benzamide;
(−)-3-{1-[3-(1-Hydroxymethyl-cyclopentyl)-propyl]-trans-3,4-dimethyl-piperidin-4-yl}-benzamide;
(+)-3-[1-(2-Hydroxy-indan-2-ylmethyl)-trans-3,4-dimethyl-piperidin-4-yl]-benzamide;
(+)-3-[1-(2-Hydroxy-indan-2-ylmethyl)-trans-3,4-dimethyl-piperidin-4-yl]-benzamide mesylate;
(+)-(3-{1-[2-(2-Hydroxy-indan-2-yl)-ethyl]-trans-3,4-dimethyl-piperidin-4-yl}-benzamide;
(+)-3-(1-{2-[3-(1-Hydroxy-cyclohexyl)-phenyl]-ethyl}trans-3,4-dimethyl-piperidin-4-yl)-benzamide;
(+)-3-[1-(cis-1-Hydroxy-3-phenyl-cyclobutylmethyl)-trans-3,4-dimethyl-piperidin-4-yl]-benzamide;
(+)-2-{2-[4-(3-Carbamoyl-phenyl)-trans-3,4-dimethyl-piperidin-1-yl]-ethyl}indan-2-carboxylic acid amide;
(+)-3-{trans-3,4-Dimethyl-1-[3-(2-nitro-indan-2-yl)-propyl]-piperidin-4-yl}-benzamide;
(+)-3-{1-[3-(2-Amino-indan-2-yl)-propyl]-trans-3,4-dimethyl-piperidin-4-yl}-benzamide;
(+)-3-{1-[cis-3-(4-Bromo-phenyl)-1-hydroxy-cyclobutylmethyl]-trans-3,4-dimethyl-piperidin-4-yl}-benzamide;
(+)-3-{1-[cis-1-Hydroxy-3-(4-methoxy-phenyl)cyclobutylmethyl]-trans-3,4-dimethyl-piperidin-4-yl-}benzamide;
(+)-31-{1-[2-(2-Amino-indan-2-yl)ethyl]-trans-3,4-dimethyl-piperidin-4-yl}-benzamide;
(+)-3-{1-[2-(2-Acetylamino-indan-2-yl)-ethyl]-trans-3,4-dimethyl-piperidin-4-yl}-benzamide;
(+)-2-{2-[4-(3-Carbamoyl-phenyl)-trans-3,4-dimethyl-piperidin-1-yl]-ethyl}-indan-2-carboxylic acid or a pharmaceutically acceptable salt of any of the above-listed compounds.

19. (+/−)-3-(trans-3,4-Dimethyl-1-phenethyl-piperidin-4-yl)-benzamide;
(+/−)-3-(1-Indan-2-ylmethyl-trans-3,4-dimethyl-piperidin-4-yl)-benzamide;
(+/−)-3-{1-[3-(1-Hydroxy-cyclohexyl)-propyl]-trans-3,4-dimethyl-piperidin-4-yl}benzamide;
(+/−)-3-{trans-3,4-Dimethyl-1-[2-(3-trifluoromethyl-phenyl)-ethyl]-piperidin-4-yl}-benzamide;
(+/−)-3-{1-[2-(4-Cyano-phenyl)-ethyl]-trans-3,4-dimethyl-piperidin-4-yl}-benzamide;
(+/−)-3-[1-(2-Hydroxy-2-phenyl-ethyl)-trans-3,4-dimethyl-piperidin-4-yl]-benzamide;
(+/−)-3-{1-[3-(1-Hydroxymethyl-cyclopentyl)-propyl]-trans-3,4-dimethyl-piperidin-4-yl}-benzamide;
(+)-3-{1-[3-(1-Hydroxymethyl-cyclopentyl)-propyl]-trans-3,4-dimethyl-piperidin-4-yl}-benzamide;
(+)-3-{1-[3-(1-Hydroxy-cyclohexyl)-propyl]-trans-3,4-dimethyl-piperidin-4-yl}-benzamide;
(−)-3-{1-[3-(1-Hydroxy-cyclohexyl)-propyl]-trans-3,4-dimethyl-piperidin-4-yl}-benzamide;
(+)-3-[1-(2-Hydroxy-indan-2-ylmethyl)-trans-3,4-dimethyl-piperidin-4-yl]-benzamide;
(+)-3-[1-(2-Hydroxy-indan-2-ylmethyl)-trans-3,4-dimethyl-piperidin-4-yl]-benzamide mesylate;
(+)-3-{1-[2-(2-Hydroxy-indan-2-yl)ethyl]-trans-3,4-dimethyl-piperidin-4-yl}-benzamide;
(+)-3-[1-(cis-1-Hydroxy-3-phenyl-cyclobutylmethyl)-trans-3,4-dimethyl-piperidin-4-yl]-benzamide;
(+)-3-{1-[cis-1-Hydroxy-3-(4-methoxy-phenyl)cyclobutylmethyl]-trans-3,4-dimethyl-piperidin-4-yl}-benzamide, or a pharmaceutically acceptable salt of any of the above-listed compounds.

20. A pharmaceutical composition comprising an effective amount of a compound according to any of claims 1-19 in combination with a pharmaceutically acceptable carrier, excipient or additive.

21. A method of treating in a mammal, in need thereof, a disease state, disorder or condition mediated by an opioid receptor or receptors which method comprises administering to said mammal an amount of a compound according to any of claims 1-19 effective in modulating an opioid receptor or receptors.

22. A method of treating in a mammal, in need thereof, a disease state, disorder or condition selected from the group consisting of irritable bowel syndrome, constipation, nausea, vomiting, pruritic dermatoses, psoriasis; eczema; an insect bite; an eating disorder, depression, anxiety, schizophrenia; drug addiction, an opioid overdose, sexual dysfunction, stroke, head trauma, traumatic brain injury, spinal damage, Parkinson's disease, Alzheimer's disease, age-related cognitive decline and Attention Deficit and Hyperactivity Disorder which method comprises administering to said mammal an amount of a compound according to any of claims 1-19 effective in treating said disease state, disorder or condition.

23. A method of treating in a mammal, in need thereof, a disease state, disorder or condition selected from the group consisting of irritable bowel syndrome, drug addiction, depression, anxiety, schizophrenia and eating disorders which method comprises administering to said mammal an amount of a compound according to any of claims 1-19 effective in treating said disease state, disorder or condition.

24. A method of treating in a mammal, in need thereof, a disease state, disorder or condition selected from the group consisting of allergic dermatitis, contact dermatitis, anorexia, bulimia, obesity, alcohol addiction, amphetamine addiction, cocaine addiction, morphine addiction, opium addiction, heroin addiction, erectile dysfunction and impotence, which method comprises administering to said mammal an effective amount of a compound according to any of claims 1-19 for treating said disease state, disorder or condition.

25. Use of a compound according to any of claims 1-19 in the manufacture of a medicament for the treatment of a mammal.

26. Use of a compound according to any of claims 1-19 in the manufacture of a medicament for the treatment of a mammal, in need thereof, of a disease state, disorder or condition selected from the group consisting of irritable bowel syndrome, constipation, nausea, vomiting, pruritic dermatoses, psoriasis, eczema; an insect bite; an eating disorder, depression, anxiety, schizophrenia; drug addiction, an opioid overdose, sexual dysfunction, stroke, head trauma, traumatic brain injury, spinal damage, Parkinson's disease, Alzheimer's disease, age-related cognitive decline and Attention Deficit and Hyperactivity Disorder.

27. Use of a compound according to any of claims 1-19 in the manufacture of a medicament for the treatment of a mammal, in need thereof, of a disease state, disorder or condition selected from the group consisting of allergic dermatitis, contact dermatitis, anorexia, bulimia, obesity, alcohol addiction, amphetamine addiction, cocaine addiction, morphine addiction, opium addiction, heroin addiction, erectile dysfunction and impotence.

28. A compound according to claim 1 wherein one or more atoms thereof have an atomic mass or mass number different from the atomic mass or mass number usually found in nature, or a pharmaceutically acceptable salt of such compound.

29. A method for obtaining an image of opioid receptors in a mammalian subject, which method comprises administering to said subject an amount of a compound according to claim 28, or pharmaceutically acceptable salt thereof, effective in imaging opioid receptors in said subject.
